(19) United States
(12) Patent Application Publication
(10) Pub. No.: US 2025/0003929 A1
(43) Pub. Date: Jan. 2, 2025

(54) HIGH THROUGHPUT SCREENING AND QUANTIFICATION FOR TARGET ANALYTES USING ECHO MS AND SINGLE TUBE CALIBRATION

(71) Applicant: DH TECHNOLOGIES DEVELOPMENT PTE. LTD., Singapore (SG)

(72) Inventors: Aaron Stella, Derry, MA (US); Bradley Schneider, Bradford (CA); Subhasish Purkayastha, Framingham, MA (US); Leigh Bedford, Ajax (CA)

(21) Appl. No.: 18/704,236

(22) PCT Filed: Oct. 26, 2022
(86) PCT No.: PCT/IB2022/060302

§ 371 Date: Apr. 24, 2024

Related U.S. Application Data
(60) Provisional application No. 63/271,913, filed on Oct. 26, 2021.

(51) Int. Cl. G01N 30/72; G01N 27/624; G01N 30/02; G01N 30/86; G01N 30/88; G01N 33/94

(52) U.S. Cl. CPC G01N 30/7233; G01N 27/624; G01N 30/8665; G01N 30/88; G01N 33/9473; G01N 33/9486; G01N 2030/027; G01N 2030/8813

(57) ABSTRACT

Disclosed are methods for detecting and quantifying target analytes in a sample by mass analysis that include detecting the presence of ions of the target analyte in the sample and quantifying the amount of the target analyte. The methods can include one or more standards (e.g., internal and/or external standards) that may be labelled (e.g., isotopically labelled).

### RELATED APPLICATIONS

The present patent application claims the priority benefit of U.S. Provisional Patent Application Ser. No. 63/271,913, filed Oct. 26, 2021, the content of which is hereby incorporated by reference in its entirety into this disclosure.

### BACKGROUND

Methods for screening and quantifying samples for one or more target analytes often incorporate a combination of techniques based on binding affinity (immunoassays and/or chromatography) and one or more analytical techniques that provide definitive characterization and quantification. For example, forensic and toxicology labs often combine immunoassays and liquid chromatography mass spectrometry (LC-MS) which provides for the screening (binding affinity) and subsequent analytical separation and structural identification of target analytes via mass spectrometry. While this combination of analytical techniques provides both screening and definitive testing, the techniques can be slow, cumbersome, and require significant resources (time and reagents). Even though immunoassays may provide the required sensitivity, they may lack specificity for particular target analytes and/or may be subject to cross-reactivity with other non-target substances that may be present in complex samples (e.g., biological/environmental samples). Accordingly, there is a need for alternative systems, methods and techniques that can provide for accurate and rapid high-throughput screening and quantification of target analyte(s) in samples.

### SUMMARY OF THE DISCLOSURE

In an aspect, the disclosure provides a method for detecting and quantifying a target analyte in a sample by mass analysis, the method comprising: providing the sample in a sample reservoir; ejecting a volume of the sample into a sampling interface; transferring the volume of sample from the sampling interface to a mass spectrometer for mass analysis; detecting by a first analysis by mass spectrometry the presence of ions of the specific analyte in the sample; and performing a second analysis that quantifies the amount of the specific analyte present in the sample.

In another aspect, the disclosure provides a method for detecting and quantifying a target analyte in a sample by mass analysis, the method comprising: providing a sample plate comprising a plurality of sample reservoirs, wherein at least one sample reservoir comprises at least one calibration standard for the target analyte, and at least one sample reservoir comprises the sample; acoustically ejecting the at least one sample into a mobile phase at an open port interface (OPI) using an acoustic droplet ejector (ADE); ionizing the at least one sample; and detecting by a first analysis by mass spectrometry the presence of ions of the target analyte in the sample, and when said ions of the target analyte are detected, performing a second analysis that quantifies the amount of the target analyte present in the sample.

In some embodiments, the second analysis comprises ionizing the sample to form ions of the target analyte and quantifying the ions of the target analyte by mass spectrometry.

In some embodiments, sample comprises an environmental or a biological sample.

In some embodiments, the above aspects and embodiments further comprise preparing the sample for analysis. In some embodiments, preparing the sample comprises diluting the sample, desalting the sample, or adding one or more internal standards to the sample. In some further embodiments preparing the sample comprises solid phase micro extraction (SPME).

In some embodiments, the above aspects and embodiments further comprise differential ion mobility separation (DMS).

In some embodiments, the target analyte comprises a pollutant, a toxin, a poison, a hormone, a drug, or metabolites thereof. In some further embodiments, the target analyte comprises a drug selected from controlled drug substances, performance enhancing drugs, prescription drugs, and/or drugs of abuse, or metabolites thereof. In some further embodiments, the target analyte comprises an opioid, or metabolite thereof. In yet further embodiments, the target analyte comprises fentanyl, norfentanyl, gabapentin, pregabalin, or PCP.

In some embodiments, the at least one calibration standard comprises two or more different calibration concentrations. In some further embodiments, quantifying the target analyte ions in the sample is based on a calibration curve generated from at least one calibration standard.

In some embodiments, the methods and systems in accordance with the disclosure provide a limit of quantitation (LOQ) of the target analyte that can be determined. While the LOQ for any particular target analyte can depend on a number of different factors, in some embodiments the methods and systems provide LOQs that are within ranges previously established for the target analyte, or improve upon previously established LOQs (e.g., LOQs associated with other established analytical methods such as, for example, LC/MS and LC-MS/MS). In some example embodiments, LOQs for target analytes can comprise values that may typically be less than about 100 ng/ml or less than about 10 ng/mL.

In some embodiments, the second analysis is performed by liquid chromatography mass spectrometry (LC-MS). In some further embodiments, second analysis comprises ejecting by ADE at least one calibration standard into a mobile phase capture fluid at an OPI; detecting ions of the calibration standard by mass spectrometry; generating calibration data; ejecting by ADE the sample into the mobile phase capture fluid at the OPI; and (i) detecting for the presence of target analyte ions in the sample by mass analysis, and (ii) quantifying the target analyte ions based on the calibration data.

In any of the above aspects and embodiments, the one or more calibration standard and the at least one sample can be in the same sample reservoir. In some further embodiments, the at least one sample comprises more than one internal calibration standard.

In any of the above aspects and embodiments, the method can comprise a plurality of samples. In some further embodiments, the plurality of samples can comprise a multi-well sampling plate.

In any of the above aspects and embodiments, the method can comprise ejecting sample at a rate of about one sample per second.

In another aspect, the disclosure provides systems configured for detecting and quantifying a target analyte in a sample by mass analysis and comprising an open port interface (OPI) and an acoustic droplet ejector (ADE).

Other aspects and embodiments of the disclosure will be apparent in light of the description and illustrative examples that follow.

### BRIEF DESCRIPTION OF THE DRAWINGS

FIGS. 1A-1B illustrate an open port interface (OPI) sampling interface and an acoustic droplet ejection (ADE) device in accordance with some example aspects and embodiments of the disclosure.

FIGS. 2A-2E illustrates a series of data obtained in accordance with the aspects and embodiments provided by the disclosure, including extracted ion chromatograms (XIC) (FIGS. 2A-2C) and peak analysis for target analytes (fentanyl and norfentanyl). Resulting calibration curves show good linearity and accuracy in concentration ranges over 3 orders of magnitude with acceptable R values (>99%), and good limits of quantification (LOQs), as confirmed by quality control experiments at three concentrations for each analyte (FIGS. 2D-2E).

FIGS. 3A-3B illustrates a series of calibration curves for a target analyte (fentanyl) determined at three different ejection volumes (2.5 nL, 10 nL, and 50 nL) that have good linearity in concentration ranges over 3 orders of magnitude with acceptable R values (>99%), and good limits of quantification (LOQs). Three quality control experiments to ensure accuracy are performed at each calibration volume.

FIG. 4 illustrates the extracted ion chromatograms (XIC) acquired in MRM mode with injection volumes of 50 nL for fentanyl (see, FIG. 3A) and internal standard for target analyte (fentanyl-d5). Each peak indicates an injected sample/sampling event.

FIGS. 5A-5B illustrates a series of calibration curves for a target analyte (norfentanyl) determined at three different ejection volumes (2.5 nL, 10 nL, and 50 nL) that have good linearity in concentration ranges over 3 orders of magnitude with acceptable R values (>99%), and good limits of quantification (LOQs). Three quality control experiments to ensure accuracy are performed at each calibration volume.

FIG. 6 illustrates the extracted ion chromatograms (XIC) acquired in MRM mode with injection volumes of 50 nL for norfentanyl (see, FIG. 5A) and internal standard for target analyte (norfentanyl-d5). Each peak indicates an injected sample/sampling event.

FIG. 7 illustrates XIC screening of 33 urine samples for the presence of fentanyl acquired with and without an internal standard (fentanyl-d5), in MRM mode with injection volume of 10 nL. Each peak indicates an injected sample with eight calibrators, three quality control samples, and one blank sample.

FIGS. 8A-8B illustrates the XIC for fentanyl-only acquisition and establishes the high-throughput method (FIG. 8A, total experimental run time of 11.3 seconds) correlates well with confirmatory analysis quantifying the amount of fentanyl in the 33 urine specimen as well as the calibrator, quality control, and blank samples (FIG. 8B).

FIG. 9 illustrates XIC screening of 33 urine samples for the presence of norfentanyl acquired without an internal standard, and demonstrates that the screening results and peak intensities correlate well with confirmatory analysis quantifying the amount of norfentanyl in the 33 urine samples as well as the calibrator, quality control, and blank samples.

FIG. 10 illustrates an overlay XIC and the individual XICs for gabapentin (2 runs) and internal standard ritalinic acid acquired in MRM mode with injection volumes of 50 nL. Each peak indicates an injected sample/sampling event.

FIGS. 11A-11B illustrates calibration curves for gabapentin using ratio of intensity of gabapentin to ritalinic acid (FIG. 11A) and peak area (FIG. 11B) at an ejection volume of 50 nL.

FIG. 12 illustrates XICs for gabapentin acquired with and without an internal standard (ritalinic acid), in MRM mode with injection volume of 20 nL. Each peak indicates an injected sample/sampling event.

FIG. 13 illustrates XIC screening of 20 urine samples for the presence of gabapentin with and without an internal standard (ritalinic acid), acquired in MRM mode with injection volume of 20 nL. Each peak indicates an injected sample with one calibrator and one blank sample.

FIG. 14 demonstrates that the screening results and peak intensities depicted in FIG. 13 generally trend with confirmatory definitive analysis that quantifies the amount of gabapentin in the 20 urine specimen, calibrator, and blank samples, obtained using LC-MS/MS.

FIGS. 15A-15B depict XICs from a single tube calibration for fentanyl/fentanyl-d5, monitoring 8 transitions at seven concentrations (FIG. 15B).

FIGS. 16A-16B depict XICs from a single tube calibration for norfentanyl/norfentanyl-d5, monitoring 7 transitions over six concentrations (FIG. 16B).

FIG. 17 depicts XICs from a single tube calibration for gabapentin, monitoring 6 transitions over five concentrations (inset).

FIGS. 18A-18G illustrates a series of extracted ion chromatograms (XICs) (FIGS. 18A-18C) from single tube calibration experiments (fentanyl and norfentanyl) and an overlay plot of the XICs with internal standard PCP-d5. The calibrations for fentanyl (18D, obtained using LC-MS/MS; 18E natural isotope calibration obtained on Echo MS) and norfentanyl (18F, obtained using LC-MS/MS; 18G natural isotope calibration obtained on Echo MS) show good linearity in concentration ranges over 3 orders of magnitude with acceptable R values (>99%) (FIGS. 18D-18G).

### DETAILED DESCRIPTION

It is to be understood that this disclosure is not limited to the particular methodology, protocols, and reagents described herein and as such may vary. It is also to be understood that the terminology used herein is for the purpose of describing particular embodiments only and is not intended to limit the scope of the present disclosure or the appended claims.

As used herein and in the appended claims, the singular forms “a,” “an,” and “the” include plural reference unless the context clearly indicates otherwise.

The term “about” is used in connection with a numerical value throughout the specification and the claims denote an interval of accuracy, familiar and acceptable to a person skilled in the art. In general, such an interval of accuracy is +/−10%.

Unless defined otherwise, all technical and scientific terms used herein have the same meaning as commonly understood to one of ordinary skill in the art to which this disclosure belongs.

The disclosure provides methods for measuring analyte and metabolite concentrations and can establish analytical levels and limits of detection, including, the limit of quantification (LOQ) which is a concentration measurement made in a range that provides for unbiased measurements within the acceptance criteria for the analytical method; lower limit of quantification (LLOQ) which is the lowest amount of an analyte in a sample that can be quantitatively determined with suitable precision and accuracy (bias); upper limit of quantification (ULOQ), which is the maximum analyte concentration of a sample that can be quantified with acceptable precision and accuracy (bias).

The disclosure generally relates to methods and systems that provide for rapid high-throughput screening and quantification of one or more target analytes in a sample. The methods and systems described herein advance the state of the art relating to analytical techniques, for example forensic and toxicologic analytical techniques, either by reducing the need for or eliminating techniques such as immunoassays and/or liquid chromatography when analyzing a sample for one or more target analytes of interest. The methods and systems described herein can decrease the time necessary to screen and quantify one or a plurality of target analyte(s) in samples, eliminate errors associated with the immunoassays in current use (e.g., cross-reactivity with non-target analytes), reduce waste and/or amount of reagents needed to perform the analysis, and reduce sample preparation time. The disclosed methods and systems provide for the sensitive and specific detection and quantification of one or more target analytes in a matter of seconds, compared to minutes, per individual sample.

The methods and systems can include additional features, including solid phase microextraction (SPME), differential mobility spectrometry (DMS), and/or single tube calibration methods, any or all of which can enhance sample throughput, method specificity, resolve analyte isomers, and reduce the potential for ion suppression and interferences.

Thus, in one aspect, the disclosure provides a method for detecting and quantifying a target analyte in a sample by mass analysis, the method comprising: providing the sample in a sample reservoir; ejecting a volume of the sample into a sampling interface; transferring the volume of sample from the sampling interface to a mass spectrometer for mass analysis; detecting by a first analysis by mass spectrometry the presence of ions of the specific analyte in the sample; and performing a second analysis that quantifies the amount of the specific analyte present in the sample.

In some embodiments of this aspect, the method can comprise: providing a sample plate comprising a plurality of sample reservoirs, wherein at least one sample reservoir comprises at least one calibration standard for the target analyte, and at least one sample reservoir comprises the sample; acoustically ejecting the at least one sample into a mobile phase at an open port interface (OPI) using an acoustic droplet ejector (ADE); ionizing the at least one sample; and detecting by a first analysis by mass spectrometry the presence of ions of the target analyte in the sample, and when said ions of the target analyte are detected, performing a second analysis that quantifies the amount of the target analyte present in the sample.

The methods can provide for contacting one or more samples-which may be unprocessed, raw, or crude samples, or may be samples that are pre-processed through one or more preparative steps such as with a solid phase microextraction (SPME) substrate under conditions effective to bind one or more target analyte in the sample. Thus, the systems and methods in accordance with various aspects and embodiments of the disclosure provide for analysis of samples that can comprise complex matrices (e.g., biological, environmental, and food samples), without extensive pre-treatment steps prior to detection and analysis of the target analyte(s) (e.g., sampling (i.e., sample collection) and sample preparation (separation from the matrix, concentration, fractionation, derivatization, etc.). In some example embodiments, the disclosure comprises solid-phase microextraction (SPME), as generally known in the art, and which can provide one or more advantages to the methods disclosed herein (e.g., integrate sampling, sample preparation, and extraction into a single step). In some non-limiting embodiments, SPME devices comprise a substrate surface that comprises a liquid or sorbent coating, as generally known in the art and/or as described herein, that binds to, and extracts from sample matrix, one or more target analytes of interest.

In accordance with the systems and methods described herein, SPME can provide for added flexibility in sample preparation and analysis. For example, in some embodiments SPME can be performed without solvents and, in some embodiments, SPME can be performed on samples in liquid and/or gas phases. In various embodiments, SPME devices can be used for ex vivo analysis using a small amount of a collected sample (i.e., a sample aliquot), and/or be used in situ by inserting a biocompatible device directly into tissue, blood, or other biological matrix for a relatively short period of time. Thus, in some embodiments comprising SPME, the method may not require separate sample collection.

In some embodiments after extraction, the SPME substrate can be stored for subsequent analysis or sample preparation. In some embodiments, desorption of the analyte(s) from the SPME substrate is performed prior to analysis. In some embodiments, the SPME substrate can be transferred to a container (e.g., sample reservoir, sampling plate, etc. for sample preparation). In some embodiments the SPME substrate can be transferred to an injection/sampling port of an instrument. In some further embodiments, the transfer may be to an open injection and/or sampling port in direct fluidic communication with an analytical instrument (e.g., mass spec) and/or an instrument that may provide for further separation of the sample components (e.g., chromatographic separations), which may also be coupled to an analytical instrument (e.g., mass spec).

In accordance with the aspect and embodiments of the disclosure, the desorption solvent used to extract sample (e.g., target analyte(s)) is effective to desorb one or more target analytes of interest from the SPME substrate. In some embodiments, the desorption solvent comprises an amount of an organic solvent or combination of organic solvents. In some embodiments the desorption solvent comprises at least about 50% (by weight or volume, e.g., from about 50%-100%) of the desorption solvent (e.g., about 50%, 55%, 60%, 65%, 70%, 75%, 80%, 85%, 90%, 95%, or 100%). Any SPME substrate (e.g., fibers, blades, micro-tips, pins, or mesh) and/or coating chemistries (e.g., polymers and solid materials that may include polydimethylsiloxanes, divinylbenzenes, carbon molecular sieve type materials (e.g., Carboxens), polyacrylates, polyethylene glycols, HLB-PAN, C18-PAN, antibodies, etc., and/or combinations thereof) known in the art or hereafter developed can be used in in accordance with the disclosure. In some embodiments, the coatings and/or substrate can be selected to improve sensitivity for the specific analytes of interest (e.g., high affinity for the target analyte(s)), and/or sized as appropriate for the sampling platform and/or analytical techniques used in the method.

In accordance with the embodiments described above, the SPME device may be fluidly coupled to an ion source for ionizing the one or more analyte species desorbed into the desorption solvent for subsequent mass spectrometric analysis (e.g., without a liquid chromatography (LC) column between the sampling interface and the ion source). In some embodiments, a substrate sampling probe (e.g., an open port interface) can be configured with a SPME device-receiving port. In accordance with such embodiments, the configuration of the sampling interface can be optimized so as to reduce the fluid volume dead space at the fluid inlet and the SPME device, which can concentrate the target analyte(s) desorbed from the SPME device in a decreased volume of the desorption solvent when the SPME device is inserted into sampling interface.

In some embodiments, the methods in accordance with the disclosure can further comprise one or more steps including, for example, conditioning the SPME substrate prior to contacting with sample, inserting the SPME substrate into sample to adsorb one or more target analyte species within the sample to the coated surface, extracting the one or more analyte species from the sample, and/or rinsing the SPME substrate (e.g., with water) prior to inserting the SPME substrate into the sample reservoir, sampling platform, and/or substrate sampling probe.

In non-limiting embodiments, the disclosure provides methods and systems comprising an ADE and an OPI (or an essentially equivalent, “open port sampling interface (OPSI)”) in fluid communication with an analytical instrument such as, e.g., a mass spectrometer. In such embodiments, the systems and methods can further comprise SPME, as described above, and which can provide the advantages of sample preparation/chromatographic-like separation that can reduce or minimize ion suppression and interference, while eliminating the need for a separate chromatographic system (either separate from or integrated into) in the system. As used herein with reference to embodiments relating to mass spectrometry, “ion suppression” and “ion interference” refer to reduced signal to noise ratio (S/N) due to ionization competition between target analytes in a sample and non-target species from endogenous or exogenous sources that are not removed from the sample matrix. In some embodiments, the methods disclosed herein reduce or minimize the co-elution of non-target species with a target analyte(s) to improve precision, accuracy, and sensitivity of an assay such as, for example, a mass spectrometry assay.

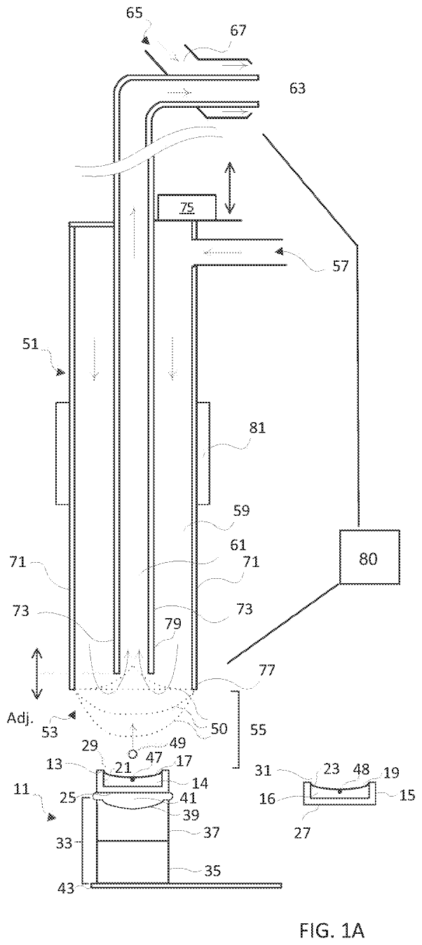

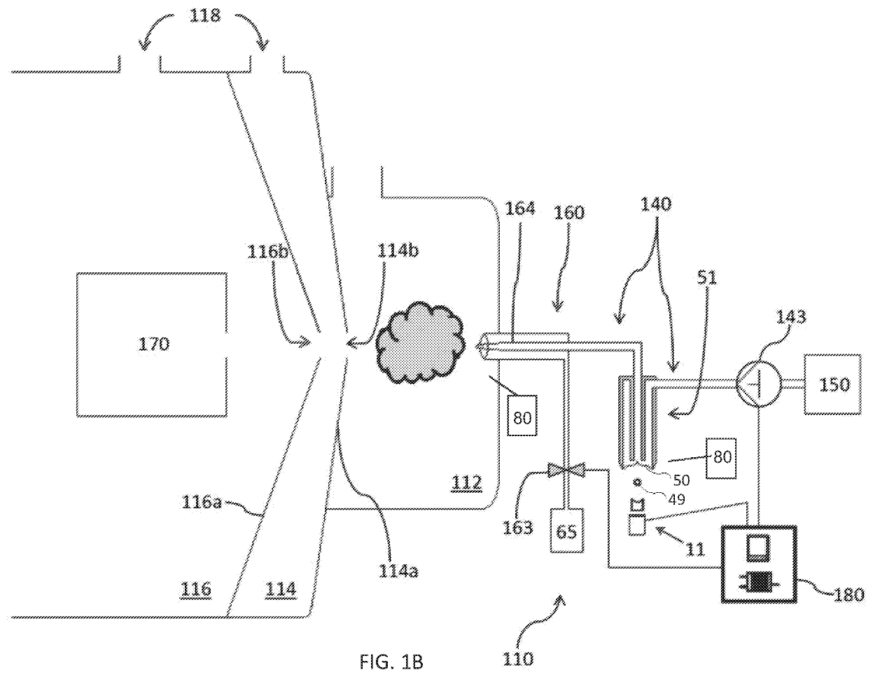

A representative system in accordance with example aspects and embodiments of the disclosure is illustrated in FIG. 1A. As with all figures referenced herein, in which like parts are referenced by like numerals, FIG. 1A is not to scale, and certain dimensions are exaggerated for clarity of presentation. In FIG. 1A, the acoustic droplet ejection (ADE) device is shown generally at 11, ejecting droplet 49 toward the continuous flow sampling probe (referred to herein as an open port interface (OPI)) indicated generally at 51 and into the sampling tip 53 thereof.

The acoustic droplet ejection device 11 includes at least one reservoir, with a first reservoir shown at 13 and an optional second reservoir 31. In some embodiments a further plurality of reservoirs may be provided. Each reservoir is configured to house a fluid sample having a fluid surface, e.g., a first fluid sample 14 and a second fluid sample 16 having fluid surfaces respectively indicated at 17 and 19. When more than one reservoir is used, as illustrated in FIG. 1A, the reservoirs are preferably both substantially identical and substantially acoustically indistinguishable, although identical construction is not a requirement.

The ADE comprises acoustic ejector 33, which includes acoustic energy generator 35 and focusing means 37 for focusing the acoustic energy generated at a focal point 47 within the fluid sample, near the fluid surface. As shown in FIG. 1A, the focusing means 37 may comprise a single solid piece having a concave surface 39 for focusing the acoustic energy, but the focusing means may be constructed in other ways as discussed below. The acoustic ejector 33 is thus adapted to generate and focus acoustic energy so as to eject a droplet of fluid from each of the fluid surfaces 17 and 19 when acoustically coupled to reservoirs 13 and 15, and thus to fluids 14 and 16, respectively. The acoustic energy generator 35 and the focusing means 37 may function as a single unit controlled by a single controller, or they may be independently controlled, depending on the desired performance of the device.

The acoustic droplet ejector 33 may be in either direct contact or indirect contact with the external surface of each reservoir. With direct contact, in order to acoustically couple the ejector to a reservoir, it is preferred that the direct contact be wholly conformal to ensure efficient acoustic energy transfer. That is, the ejector and the reservoir should have corresponding surfaces adapted for mating contact. Thus, if acoustic coupling is achieved between the ejector and reservoir through the focusing means, it is desirable for the reservoir to have an outside surface that corresponds to the surface profile of the focusing means. Without conformal contact, efficiency and accuracy of acoustic energy transfer may be compromised. In addition, since many focusing means have a curved surface, the direct contact approach may necessitate the use of reservoirs that have a specially formed inverse surface.

Optimally, acoustic coupling is achieved between the ejector and each of the reservoirs through indirect contact, as illustrated in FIG. 1A. In the figure, an acoustic coupling medium 41 is placed between the ejector 33 and the base 25 of reservoir 13, with the ejector and reservoir located at a predetermined distance from each other. The acoustic coupling medium may be an acoustic coupling fluid, preferably an acoustically homogeneous material in conformal contact with both the acoustic focusing means 37 and the underside of the reservoir. In addition, it is important to ensure that the fluid medium is substantially free of material having different acoustic properties than the fluid medium itself. As shown, the first reservoir 13 is acoustically coupled to the acoustic focusing means 37 such that an acoustic wave generated by the acoustic energy generator is directed by the focusing means 37 into the acoustic coupling medium 41, which then transmits the acoustic energy into the reservoir 13.

In operation, reservoir 13 and optional reservoir 15 of the device are filled with first and second fluid samples 14 and 16, respectively, as shown in FIG. 1A. The acoustic ejector 33 is positioned just below reservoir 13, with acoustic coupling between the ejector and the reservoir provided by means of acoustic coupling medium 41. Initially, the acoustic ejector is positioned directly below sampling tip 53 of OPI 51, such that the sampling tip faces the surface 17 of the fluid sample 14 in the reservoir 13. Once the ejector 33 and reservoir 13 are in proper alignment below sampling tip 53, the acoustic energy generator 35 is activated to produce acoustic energy that is directed by the focusing means 37 to a focal point 47 near the fluid surface 17 of the first reservoir. As a result, droplet 49 is ejected from the fluid surface 17 toward and into the liquid boundary 50 at the sampling tip 53 of the OPI 51, where it combines with solvent in the flow probe 53. The profile of the liquid boundary 50 at the sampling tip 53 may vary from extending beyond the sampling tip 53 to projecting inward into the OPI 51. In a multiple-reservoir system, the reservoir unit (not shown), e.g., a multi-well plate or tube rack, can then be repositioned relative to the acoustic ejector such that another reservoir is brought into alignment with the ejector and a droplet of the next fluid sample can be ejected. The solvent in the flow probe cycles through the probe continuously, minimizing or even eliminating “carryover” between droplet ejection events. Fluid samples 14 and 16 are samples of any fluid for which transfer to an analytical instrument is desired, where the term “fluid” is as defined earlier herein.

The structure of OPI 51 is also shown in FIG. 1A. Any number of commercially available continuous flow sampling probes can be used as is or in modified form, all of which, as is well known in the art, operate according to substantially the same principles. As can be seen in the FIG. 1A, the sampling tip 53 of OPI 51 is spaced apart from the fluid surface 17 in the reservoir 13, with a gap 55 therebetween. The gap 55 may be an air gap, or a gap of an inert gas, or it may comprise some other gaseous material; there is no liquid bridge connecting the sampling tip 53 to the fluid 14 in the reservoir 13. The OPI 51 includes a solvent inlet 57 for receiving solvent from a solvent source and a solvent transport capillary 59 for transporting the solvent flow from the solvent inlet 57 to the sampling tip 53, where the ejected droplet 49 of analyte-containing fluid sample 14 combines with the solvent to form an analyte-solvent dilution. A solvent pump (not shown) is operably connected to and in fluid communication with solvent inlet 57 in order to control the rate of solvent flow into the solvent transport capillary and thus the rate of solvent flow within the solvent transport capillary 59 as well.

Fluid flow within the OPI 51 carries the analyte-solvent dilution through a sample transport capillary 61 provided by inner capillary tube 73 toward sample outlet 63 for subsequent transfer to an analytical instrument. A sampling pump (not shown) can be provided that is operably connected to and in fluid communication with the sample transport capillary 61, to control the output rate from outlet 63. In a preferred embodiment, a positive displacement pump is used as the solvent pump, e.g., a peristaltic pump, and, instead of a sampling pump, an aspirating nebulization system is used so that the analyte-solvent dilution is drawn out of the sample outlet 63 by the Venturi effect caused by the flow of the nebulizing gas introduced from a nebulizing gas source 65 via gas inlet 67 (shown in simplified form in FIG. 1A, insofar as the features of aspirating nebulizers are well known in the art) as it flows over the outside of the sample outlet 63. The analyte-solvent dilution flow is then drawn upward through the sample transport capillary 61 by the pressure drop generated as the nebulizing gas passes over the sample outlet 63 and combines with the fluid exiting the sample transport capillary 61. A gas pressure regulator is used to control the rate of gas flow into the system via gas inlet 67. In a preferred manner, the nebulizing gas flows over the outside of the sample transport capillary 61 at or near the sample outlet 63 in a sheath flow type manner which draws the analyte-solvent dilution through the sample transport capillary 61 as it flows across the sample outlet 63 that causes aspiration at the sample outlet upon mixing with the nebulizer gas.

The solvent transport capillary 59 and sample transport capillary 61 are provided by outer capillary tube 71 and inner capillary tube 73 substantially co-axially disposed therein, where the inner capillary tube 73 defines the sample transport capillary, and the annular space between the inner capillary tube 73 and outer capillary tube 71 defines the solvent transport capillary 59.

The system can also include an adjuster 75 coupled to the outer capillary tube 71 and the inner capillary tube 73. The adjuster 75 can be adapted for moving the outer capillary tube tip 77 and the inner capillary tube tip 79 longitudinally relative to one another. The adjuster 75 can be any device capable of moving the outer capillary tube 71 relative to the inner capillary tube 73. Exemplary adjusters 75 can be motors including, but are not limited to, electric motors (e.g., AC motors, DC motors, electrostatic motors, servo motors, etc.), hydraulic motors, pneumatic motors, translational stages, and combinations thereof. As used herein, “longitudinally” refers to an axis that runs the length of the probe 51, and the inner and outer capillary tubes 73, 71 can be arranged coaxially around a longitudinal axis of the probe 51, as shown in FIG. 1A-B.

Additionally, as illustrated in FIG. 1A, the OPI 51 may be generally affixed within an approximately cylindrical holder 81, for stability and ease of handling.

FIG. 1B schematically depicts an embodiment of an exemplary system 110 in accordance with various aspects of the applicant's teachings for ionizing and mass analyzing analytes received within an open end of a sampling probe 51, the system 110 including an acoustic droplet injection device 11 configured to inject a droplet 49, from a reservoir into the open end of the sampling probe 51. As shown in FIG. 1B, the exemplary system 110 generally includes a sampling probe 51 (e.g., an open port interface) in fluid communication with a nebulizer-assisted ion source 160 for discharging a liquid containing one or more sample analytes (e.g., via electrospray electrode 164) into an ionization chamber 112, and a mass analyzer 170 in fluid communication with the ionization chamber 112 for downstream processing and/or detection of ions generated by the ion source 160. A fluid handling system 140 (e.g., including one or more pumps 143 and one or more conduits) provides for the flow of liquid from a solvent reservoir 150 to the sampling probe 51 and from the sampling probe 51 to the ion source 160. For example, as shown in FIG. 1B, the solvent reservoir 150 (e.g., containing a liquid, desorption solvent) can be fluidly coupled to the sampling probe 51 via a supply conduit through which the liquid can be delivered at a selected volumetric rate by the pump 143 (e.g., a reciprocating pump, a positive displacement pump such as a rotary, gear, plunger, piston, peristaltic, diaphragm pump, or other pump such as a gravity, impulse, pneumatic, electrokinetic, and centrifugal pump), all by way of non-limiting example. As discussed in detail below, flow of liquid into and out of the sampling probe 51 occurs within a sample space accessible at the open end such that one or more droplets can be introduced into the liquid boundary 50 at the sample tip 53 and subsequently delivered to the ion source 160. As shown, the system 110 includes an acoustic droplet injection device 11 that is configured to generate acoustic energy that is applied to a liquid contained with a reservoir (as depicted in FIG. 1A) that causes one or more droplets 49 to be ejected from the reservoir into the open end of the sampling probe 51. A controller 180 can be operatively coupled to the acoustic droplet injection device 11 and can be configured to operate any aspect of the acoustic droplet injection device 11 (e.g., focusing means, acoustic radiation generator, automation means for positioning one or more reservoirs into alignment with the acoustic radiation generator, etc.) so as to inject droplets into the sampling probe 51 or otherwise discussed herein substantially continuously or for selected portions of an experimental protocol by way of non-limiting example.

As shown in FIG. 1B, the exemplary ion source 160 can include a source 65 of pressurized gas (e.g. nitrogen, air, or a noble gas) that supplies a high velocity nebulizing gas flow which surrounds the outlet end of the electrospray electrode 164 and interacts with the fluid discharged therefrom to enhance the formation of the sample plume and the ion release within the plume for sampling by 114b and 116b, e.g., via the interaction of the high speed nebulizing flow and jet of liquid sample (e.g., analyte-solvent dilution). The nebulizer gas can be supplied at a variety of flow rates, for example, in a range from about 0.1 L/min to about 20 L/min, which can also be controlled under the influence of controller 180 (e.g., via opening and/or closing valve 163). In accordance with various aspects of the present teachings, it will be appreciated that the flow rate of the nebulizer gas can be adjusted (e.g., under the influence of controller 180) such that the flow rate of liquid within the sampling probe 51 can be adjusted based, for example, on suction/aspiration force generated by the interaction of the nebulizer gas and the analyte-solvent dilution as it is being discharged from the electrospray electrode 164 (e.g., due to the Venturi effect).

In the depicted embodiment, the ionization chamber 112 can be maintained at an atmospheric pressure, though in some embodiments, the ionization chamber 112 can be evacuated to a pressure lower than atmospheric pressure or maintained at a pressure higher than atmospheric pressure. The ionization chamber 112, within which the analyte can be ionized as the analyte-solvent dilution is discharged from the electrospray electrode 164, is separated from a gas curtain chamber 114 by a plate 114a having a curtain plate aperture 114b. As shown, a vacuum chamber 116, which houses the mass analyzer 170, is separated from the curtain chamber 114 by a plate 116a having a vacuum chamber sampling orifice 116b. The curtain chamber 114 and vacuum chamber 116 can be maintained at a selected pressure(s) (e.g., the same or different sub-atmospheric pressures, a pressure lower than the ionization chamber) by evacuation through one or more vacuum pump ports 118.

It will also be appreciated by a person skilled in the art and in light of the teachings herein that the mass analyzer 170 can have a variety of configurations. Generally, the mass analyzer 170 is configured to process (e.g., filter, sort, dissociate, detect, trap, etc.) sample ions generated by the ion source 160. By way of non-limiting example, the mass analyzer 170 can be a triple quadrupole mass spectrometer, or any other mass analyzer known in the art and modified in accordance with the teachings herein. Other non-limiting, exemplary mass spectrometer systems that can be modified in accordance various aspects of the systems, devices, and methods disclosed herein can be found, for example, in an article entitled “Product ion scanning using a Q-q-Qₗᵢₙₑₐᵣ ion trap (Q TRAP®) mass spectrometer,” authored by James W. Hager and J. C. Yves Le Blanc and published in Rapid Communications in Mass Spectrometry (2003; 17:1056-1064), and U.S. Pat. No. 7,923,681, entitled “Collision Cell for Mass Spectrometer,” which are hereby incorporated by reference in their entireties. Other configurations, including but not limited to those described herein and others known to those skilled in the art, can also be utilized in conjunction with the systems, devices, and methods disclosed herein. For instance other suitable mass spectrometers include single quadrupole, triple quadrupole, ToF, trap, and hybrid analyzers. It will further be appreciated that any number of additional elements can be included in the system 110 including, for example, an ion mobility spectrometer (e.g., a differential mobility spectrometer for ion selection) that is disposed between the ionization chamber 112 and the mass analyzer 170 and is configured to separate ions based on their mobility through a drift gas in high- and low-fields rather than their mass-to-charge ratio). Additionally, it will be appreciated that the mass analyzer 170 can comprise a detector that can detect the ions which pass through the analyzer 170 and can, for example, supply a signal indicative of the number of ions per second that are detected. It will also be apparent to those of skill in the relevant arts that mass analyzer 170 may include a series of differentially pumped vacuum chambers with one or more ion guides (not shown).

In accordance with the aspect and embodiments of the disclosure, an acoustic signal can be detected and/or monitored in one or more regions of the sampling system. In example embodiment in accordance with FIGS. 1A-1B the signal may be detected and/or monitored by a detection device or acoustic transducer 80, at or near the sampling interface region generally in the area of the liquid boundary 50, sampling tip 53, and/or gap 55, at a receiving end of a sample transport capillary (e.g., 61, 73, 77, 79) and/or at the opposite end of the fluid handling system (e.g., outlet 63 of transport capillary in FIG. 1A, 140 in FIG. 1B) that transfers sample to a secondary device such as an ionization chamber 112, an ion source 160, and/or an electrospray electrode 164 (e.g., as depicted in the example embodiment of FIG. 1B).

In some embodiments, the methods can include differential mobility spectrometry (DMS), which may also be referred to as Field Asymmetric Waveform Ion Mobility Spectrometry (FAIMS) or Field Ion Spectrometry (FIS). DMS typically performs gas-phase ion sample separation and analysis by continuously transmitting ions-of-interest while filtering out unwanted/non-selected species. In accordance with the example aspects and embodiments of the disclosure, a DMS can be interfaced with a mass spectrometer (MS (e.g., an ADE/OPI MS)) to take advantage of the atmospheric pressure, gas-phase, and continuous ion separation capabilities of the DMS and the detection accuracy of the MS. The combination of a DMS with an MS has enhanced numerous areas of complex sample analysis, including proteomics, peptide/protein conformation, pharmacokinetics, and metabolic processes. In addition to pharmaceutical and biotech applications, DMS-based analyzers have been used for trace level explosives detection and petroleum monitoring.

A DMS separates and analyzes ions based on the mobility characteristics of the ions rather than based on the mass-to-charge ratio as in MS. Specifically in DMS, ions within a drift gas can be continuously sampled, between two parallel electrodes that generate an asymmetric electric field (S or separation field) therebetween that tends to move the ions in a direction perpendicular to the direction of the drift gas flow (i.e., toward the electrodes). The asymmetric field(S) can be generated by applying an electrical signal(s) (e.g., RF voltages) to one or more of the electrodes so as to generate an asymmetric waveform, the amplitude of which is referred to as the SV (separation voltage). Typically, the DMS is in fluid communication with a mass spectrometer in any variety of configurations that are generally known and described in the art (see, e.g., U.S. Pat. No. 8,084,736, US2019/0113478 and US2019/0086363 all of which are incorporated herein by reference). Suitably, the DMS is configured to resolve ions (e.g., ionized isobaric species) based on their mobility through a fixed or variable electric field. As such, the DMS can comprise any ion mobility device configured to separate ions based on their mobility through a carrier or drift gas, including by way of non-limiting example, an ion mobility spectrometer, a drift-time ion mobility spectrometer, a traveling-wave ion mobility spectrometer, a differential mobility spectrometer, and a high-field asymmetric waveform ion mobility spectrometer (FAIMS) of various geometries such as parallel plate, curved electrode, spherical electrode, micromachined FAIMS, or cylindrical FAIMS device, among others.

In some embodiments, the systems and methods comprise a sampling platform that comprises a sample reservoir or a plurality of reservoirs (e.g., sample well plate) operatively configured and/or addressable to an ADE. In some embodiments a plurality of sample reservoirs are prepared with each reservoir comprising a sample and a concentration of internal standard. In some embodiments a plurality of sample reservoirs are prepared with each reservoir comprising a sample solution and an optional concentration of internal standard. In some embodiments a series of sample reservoirs are prepared wherein any reservoir that comprises a sample does not comprise an internal standard, and wherein a separate reservoir (i.e., a reservoir that does not comprise a sample) comprises a concentration of calibration standard. Volumes from each of the reservoirs can be analyzed for the presence or absence of one or more components (e.g., target analyte(s)) in the sample.

In some embodiments the methods relate to one or more samples comprising a known amount of one or more compounds as internal standards. In such embodiments, the one or more compounds may be the same as the target analyte(s). In further embodiments, the one or more compounds may comprise a label such as, for example, one or more isotopic labels (e.g., deuterium (D or ²H)-labelled, ¹³C-labelled, ¹⁵N-labelled, ¹⁷O- and ¹⁸O-labelled, ³⁵Cl and ³⁷Cl-labelled, etc.) that may be used, e.g., for the quantification of target analyte. In embodiments relating to isotopic labelling, the isotopically-enriched compound may comprise a plurality of atoms comprising the isotope and/or may comprise high isotopic purity. When combined with multiple reaction monitoring (MRM) (or alternatively “selected reaction monitoring” (SRM)), such methods can allow for detection and quantification of target analytes at low concentrations in body fluids.

In some embodiments, the methods provide for external calibration standards that are prepared as a mixture of standards in a single tube or reservoir. In some embodiments the single tube calibrator comprises a mixture of isotopologues of one or more target analytes or metabolites thereof.

In accordance with example embodiments of the disclosure, the second analysis for quantifying target analyte can comprise mass spec analysis that may further comprise liquid chromatography, microflow, and solid phase extraction.

In some embodiments, the disclosure provides a MS device equipped with acoustic ejection and a method that can both screen and quantify target analyte concentrations (i.e., drugs, toxins, hormones, etc.) in a single ejection (e.g., from about 2.5 nL to 50 nL, or more). The ADE can be adapted to rapidly eject nanoliter-sized droplets (e.g., at ˜1.0 sec/sample) into an OPI. The sample volumes ejected by ADE in combination with the low volume and flow rate of the capture liquid in the OPI allows for high throughput analysis wherein, e.g., a reservoir plate comprising 96 sample wells can be mass analyzed in MRM mode in approximately 2.4 minutes (0.04 hours) using approximately 1.03 mL of mobile phase. The number of sample reservoirs can be scaled up (e.g., 384- and 1536-wells) to increase throughput.

In accordance with the aspects and embodiments described herein the target analyte(s) that are detected and quantified by the systems and methods disclosed herein are present in a sample. The sample can be any sample that is amenable to analysis. In some embodiments the sample comprises a liquid sample. In some embodiments the sample comprises a biological sample. In some embodiments, the sample can include non-limiting examples of blood, plasma, serum, and other bodily fluids or excretions, (e.g, saliva, urine, cerebrospinal fluid, lacrimal fluid, perspiration, gastrointestinal fluid, amniotic fluid, mucosal fluid, pleural fluid, sebaceous oil, exhaled breath/droplets, and the like). In some aspects, the liquid sample further comprises an internal standard.

In some aspects, the methods and systems can screen for and quantify any chemical or biological target analyte of interest. In some embodiments, the target analyte(s) can comprise pollutants, toxins, poisons, hormones, drugs (controlled substances, performance enhancing drugs, etc.), metabolites, and the like. In some embodiments, the target analyte comprises a hormone or drug including for example, performance enhancing drugs (e.g., testosterone or derivatives thereof, human growth hormone, etc.), prescription drugs, or drugs of abuse (e.g., opioids (narcotics), amphetamines, methamphetamines, benzodiazepines, barbiturates, marijuana, cocaine, PCP, or methadone, and the like).

In some embodiments, the method comprises preparing a liquid sample for clinical analysis. The clinical analysis can screen for drugs of abuse. As discussed herein, some non-limiting examples of drugs of abuse include amphetamines, methamphetamines, benzodiazepines, barbiturates, marijuana, cocaine, PCP, methadone, and opioids (narcotics). In some embodiments, the clinical analysis comprises urine test or a urinalysis that screens for drugs of abuse.

The aspects and embodiments generally disclosed above can be further understood in view of the following examples which are provided merely for the purpose of illustrating some aspects and particular embodiments in accordance with the disclosure. Other aspects and embodiments in accordance with the general guidance and description provided by the disclosure will be apparent to those skilled in the art.

### EXAMPLES

### Example 1: Analysis of Target Analytes in Urine Samples

The series of experiments presented below demonstrate that high throughput mass spec analysis can rapidly and accurately screen for specific target analytes (e.g., drugs of abuse) in a biological sample (e.g., urine samples, SPME samples, etc.). As shown below, when used in combination with calibration curves for the target analyte(s), the high throughput methods can identify the presence of (i.e., rapidly screen for) target analyte(s) in samples while also providing accurate estimates of the amount of target analyte in the sample. These results can be confirmed and validated by subsequent quantitative analytic methods as described throughout the disclosure or as otherwise available and known in the art.

As illustrated below, the methods can be performed on samples that include internal standards for targeted analyte screening, or on samples that do not include internal standards. Samples without internal standards can be quantified based on an external standard (e.g., an established calibration curve), or analyzed and qualitatively estimated as performed in the initial screening analysis.

SPME Sample Preparation. Several examples incorporate SPME in order to reduce or eliminate ion suppression and interferences arising from sample matrix.

Samples for analysis are prepared from aliquots of urine samples, with or without internal standards (IS), (100 μL sample without IS, or 90 μL sample mixed with 10 μL IS, when included). The resulting sample (sample+IS, or sample alone) is contacted with an SPME substrate for about 30 min. The SPME substrate is optionally rinsed (e.g., with water) prior to extraction (˜ 5 min. mixing) in an extraction medium comprising methanol (e.g., 50-100% methanol). As an alternative, the SPME substrate can be applied directly to a sampling or injection port interface that is configured to receive the substrate, and the target analyte(s) extracted for analysis, and as generally described herein. Once extracted into extraction medium, an amount of the resulting extract can be loaded to a sample reservoir (e.g., an addressable multi-well sample plate) prior to mass analysis.

### Example 1A—Analysis of Fentanyl/Norfentanyl in Urine Matrix

A series of experiments are performed on 33 urine samples to detect and quantify fentanyl and its metabolite, norfentanyl, using Echo MS (AEMS). The results from the initial sample screening are evaluated against a series of experimentally established calibration curves, and target analyte concentrations are validated subsequently using confirmatory LC-MS/MS methods.

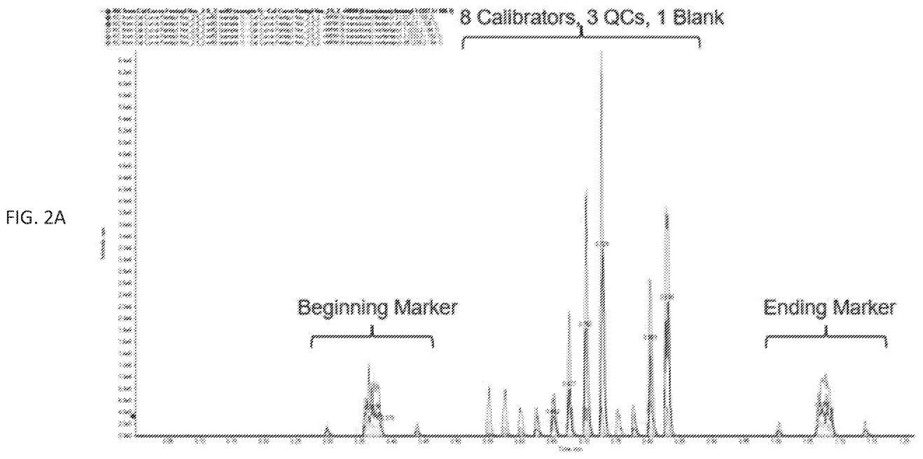

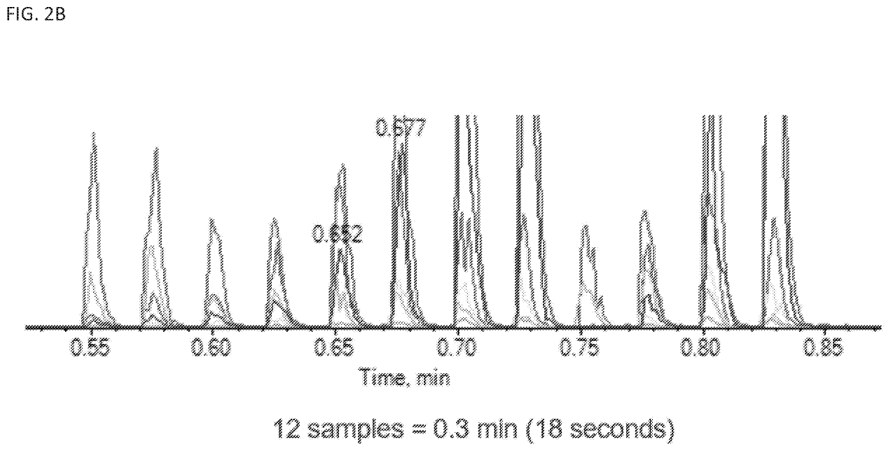

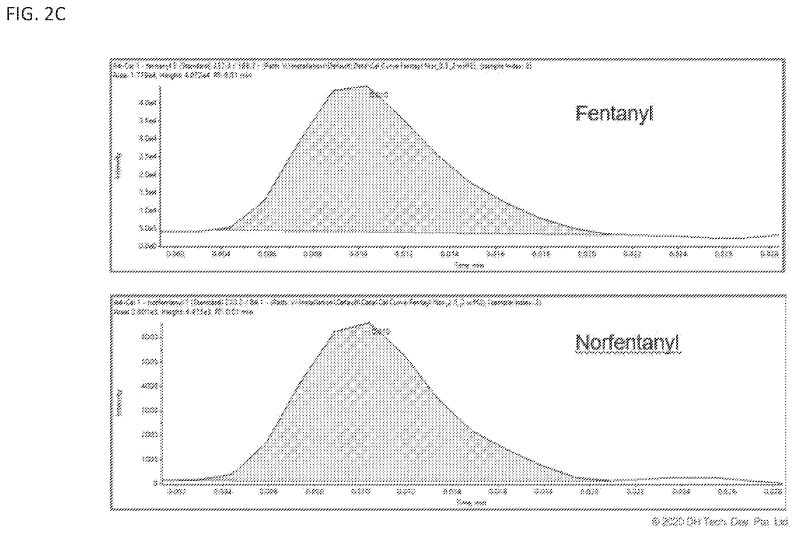

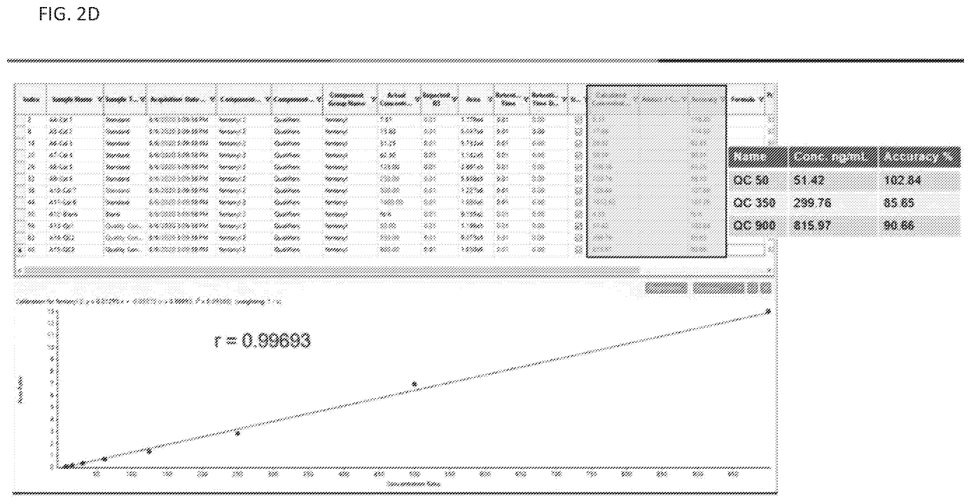

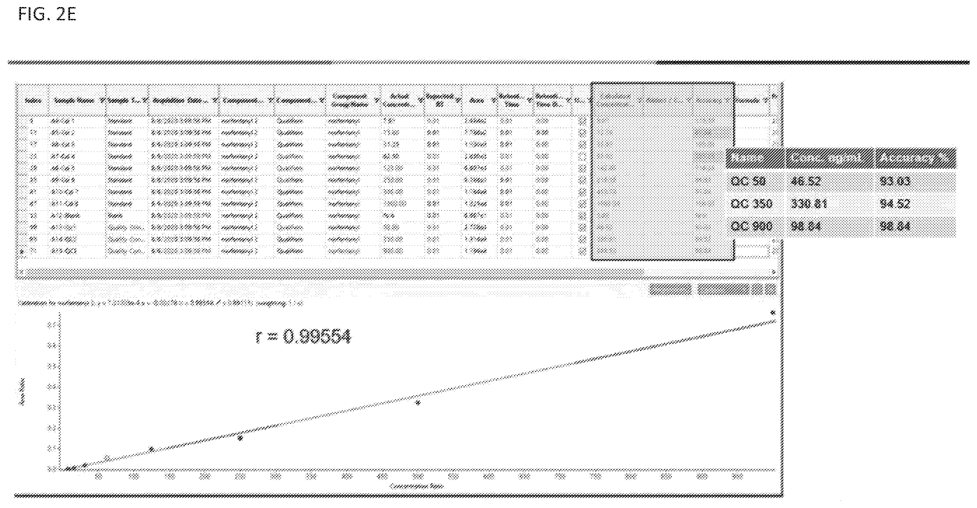

Calibration curves are established in duplicate using fentanyl and norfentanyl calibrators at 8 different concentrations (1000 ng/ml, 500 ng/mL, 250 ng/mL, 125 ng/ml, 62.5 ng/ml, 31.25 ng/mL, 15.6 ng/ml, and 7.81 ng/mL) and that include internal standards (fentanyl-d5 and norfentantyl-d5). In addition to the calibrators, the calibration curves are run with a blank sample and 3 quality controls (at 50 ng/mL, 250 ng/mL, and 900 ng/ml) for both fentanyl and norfentanyl. FIG. 2A depicts the XIC, for 6 MRMs, identifying the start and end of run markers, the 8 calibrator samples, the blank sample, and the 3 QC samples. FIG. 2B provides an expanded view of the lower intensity XICs, and indicates the analysis of the 12 samples is completed in 18 seconds. FIG. 2C illustrates peak shape and area calculation for the lowest concentrations (7.81 ng/mL). The resulting calibration curves are depicted in FIG. 2D (fentanyl) and FIG. 2E (norfentanyl).

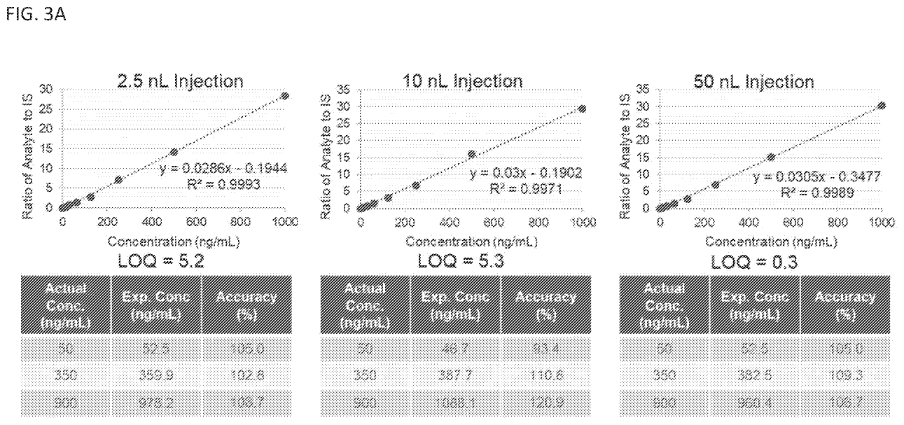

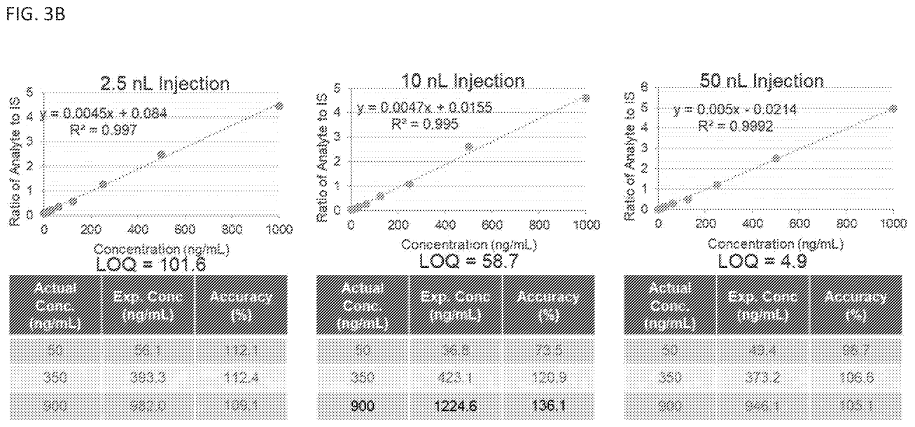

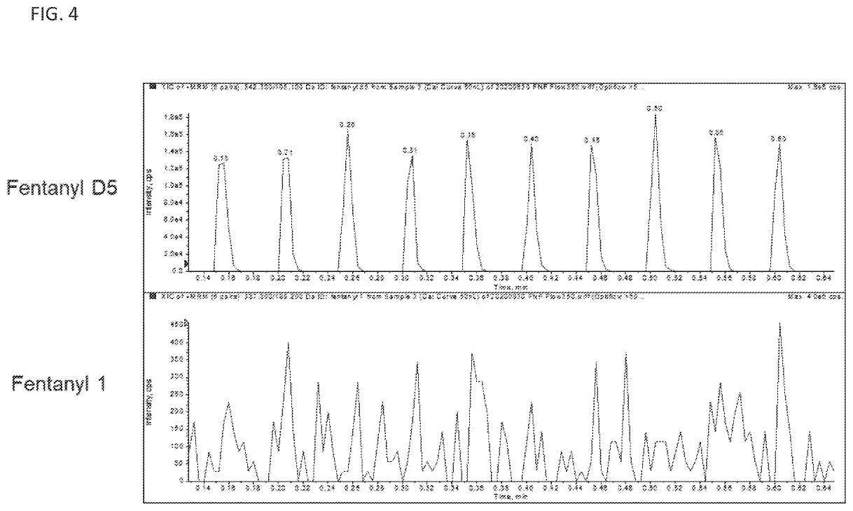

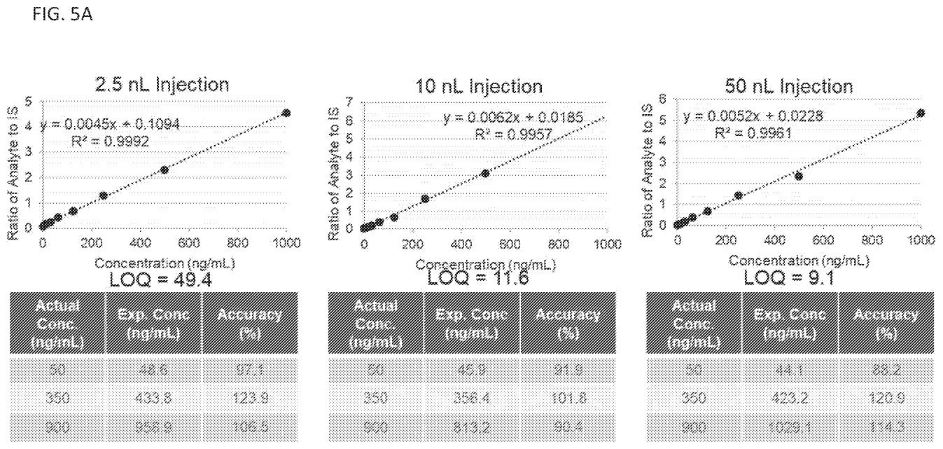

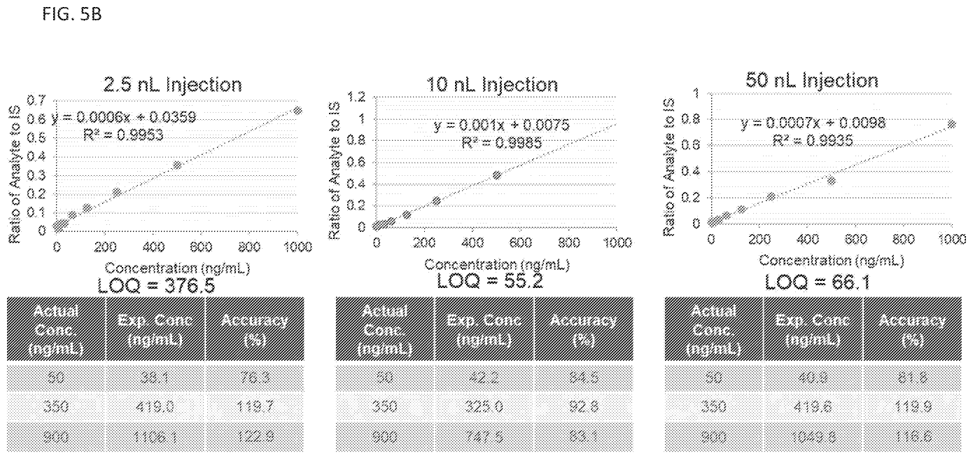

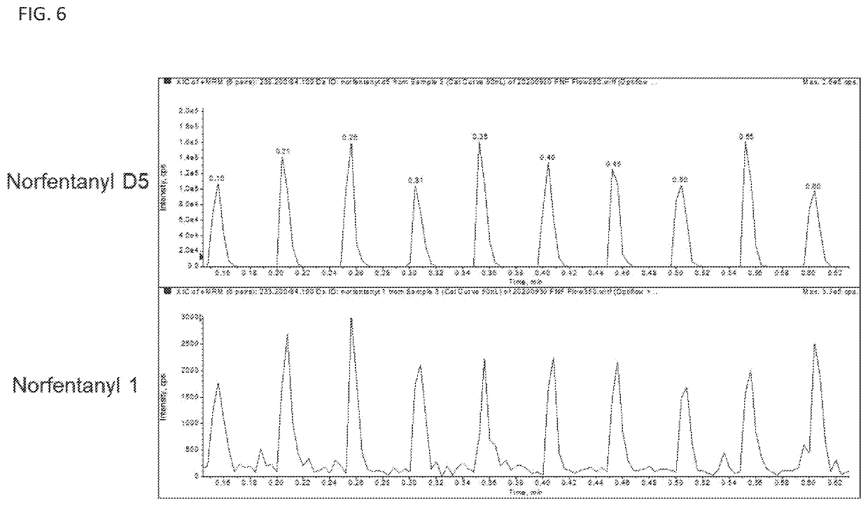

A second series of calibration curves for each of the target analytes fentanyl and norfentanyl are prepared in duplicate for injected sample volumes of 2.5 nL, 10 nL, and 50 nL, over a concentration range spanning three orders of magnitude, up to 1000 ng/ml (same concentrations as in FIGS. 2A-E). The calibration curves are prepared with internal standard for fentanyl and norfentanyl, using the isotopologues fentanyl-d5 and norfentanyl-d5, respectively and are used to determine LOQs and linear dynamic range, with the x-axis as the fentanyl/norfentanyl concentrations, and the y-axis as the peak ratio of fentanyl/fentanyl-d5 and norfentanyl/norfentanyl-d5. As illustrated by FIGS. 3A and 3B, the fentanyl calibration curves at each injection volume demonstrated good linearity and limits of quantitation (LOQs) over a wide range of concentrations. Accuracy of the calibrations were confirmed using quality control samples at 50 ng/mL, 250 ng/mL and 900 ng/mL. Similar results are obtained for norfentanyl (FIGS. 5A, 5B)

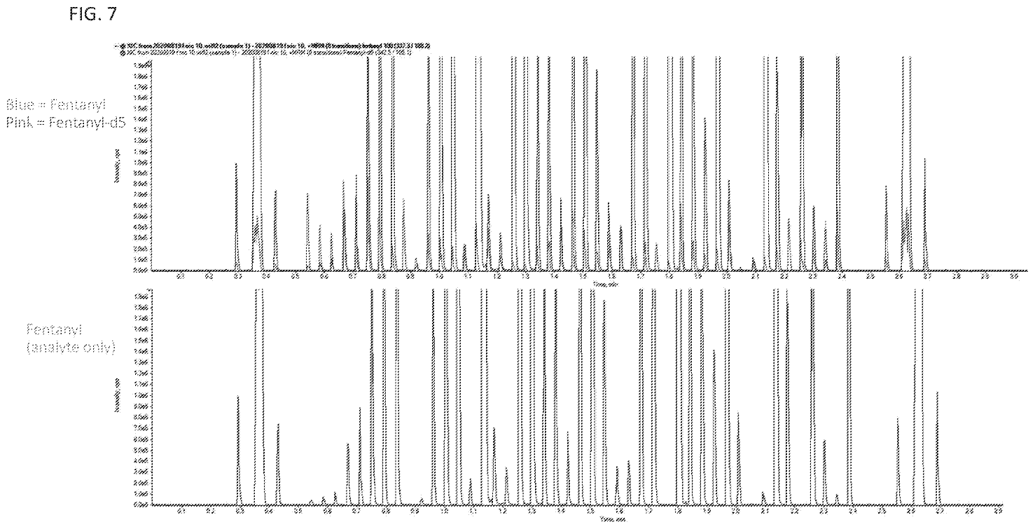

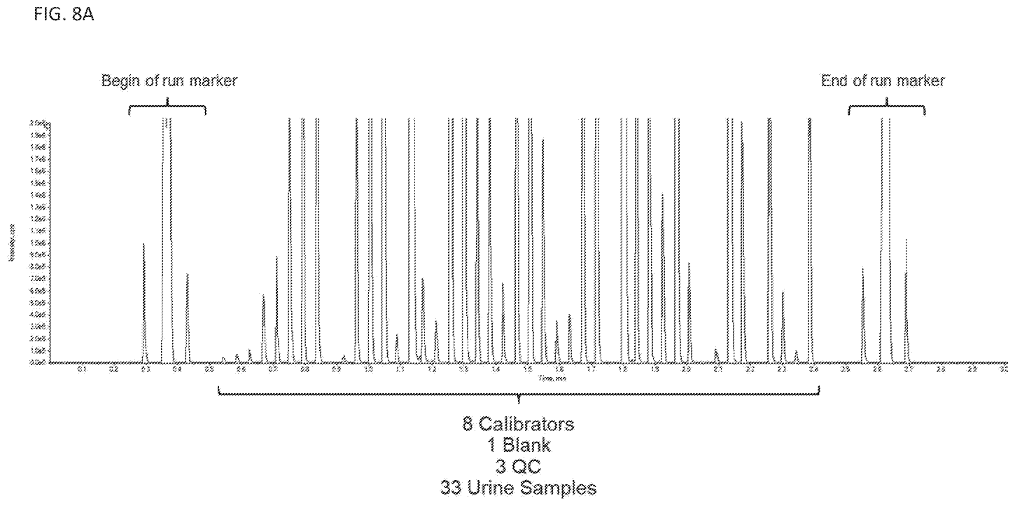

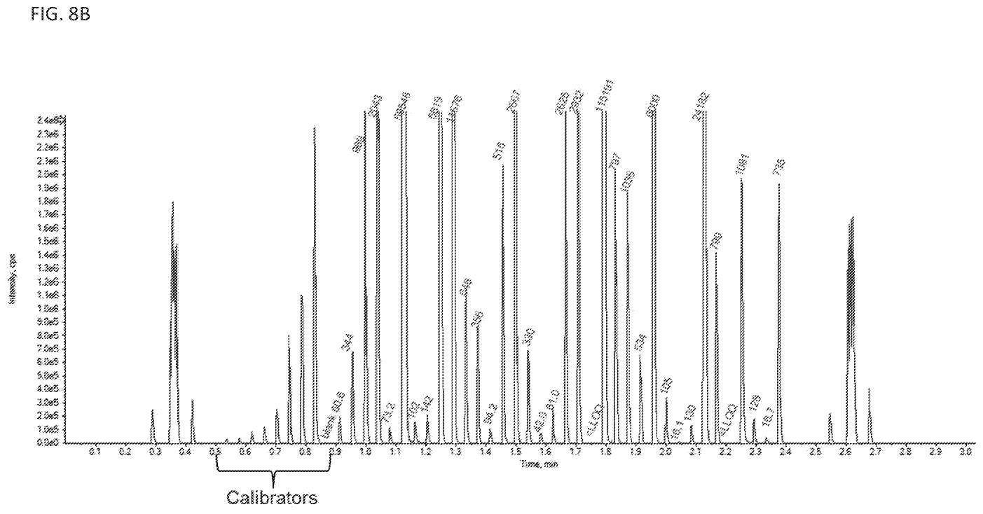

The 33 urine samples are prepared as described above. Sample droplets (10 nL) are ejected by ADE into the open port interface operably connected to an ESI ion source, operated in MRM mode (342.5/105.1 (fentanyl-d5 detection) and 337.3/188.2 for fentanyl analysis; 236.2/86.1 (norfentanyl-d5 detection) and 238.2/84.1 for norfentanyl analysis). Total run time between the start of run and end of run markers is 111.3 seconds. See, FIGS. 7, 8A.

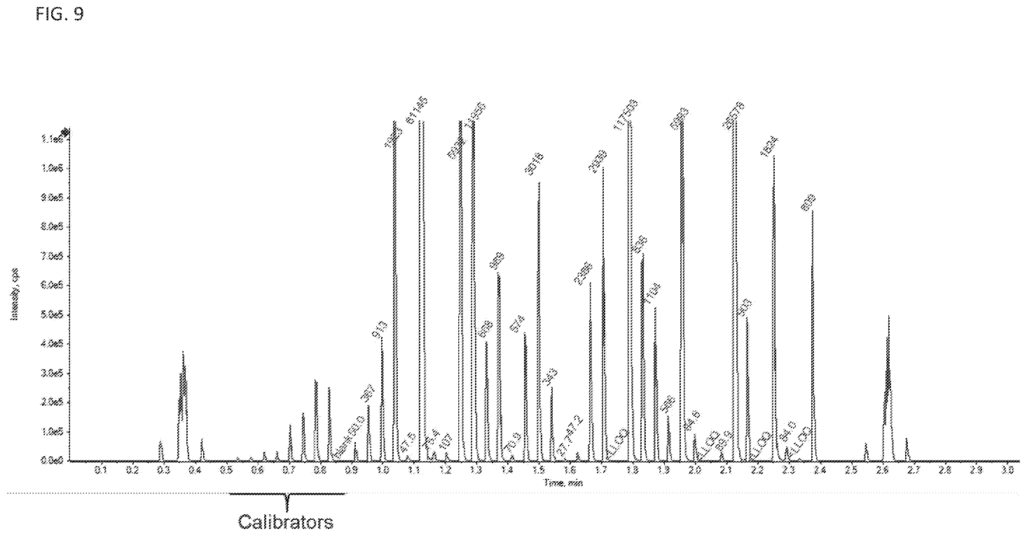

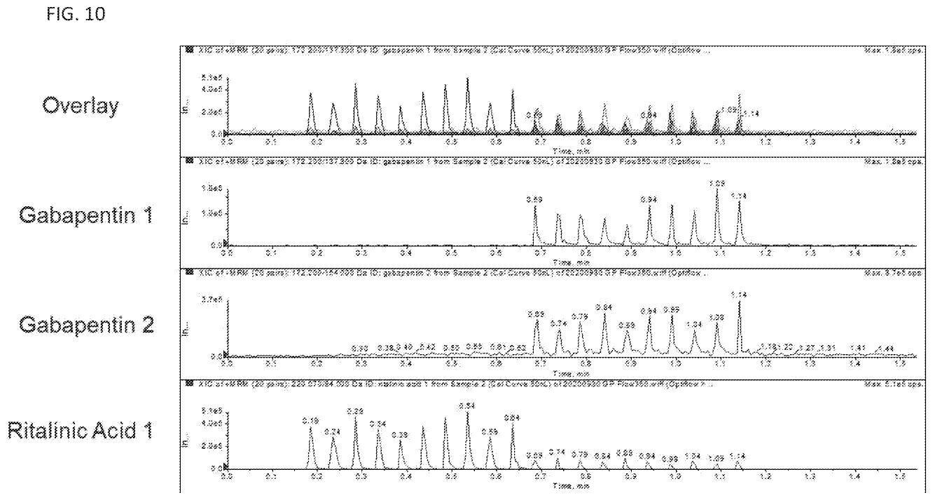

Based on the XIC intensities and established calibration curves, an estimation of fentanyl/norfentanyl concentration in each sample can be made. Further confirmatory definitive testing of each urine sample is made by LC/MS using a Microflow M5 column (50×1 mm, 2.6 um) on a 6500+ QTRAP (Sciex). The definitive analytical testing confirms that the high throughput analytical methods provide good estimates of target analyte concentrations relative to definitive testing reference methods, as shown in FIGS. 8B and 9.

### Example 1B—Analysis of Gabapentin in Urine Matrix

A series of experiments are performed on 20 urine samples to detect and quantify gabapentin using Echo MS (AEMS). The results from the initial sample screening are evaluated against a series of experimentally established calibration curves, and target analyte concentrations are validated subsequently using confirmatory LC-MS/MS methods.

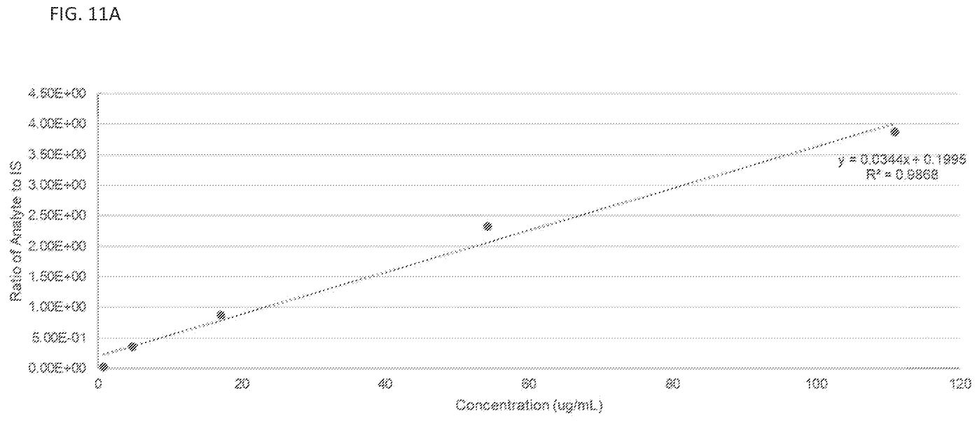

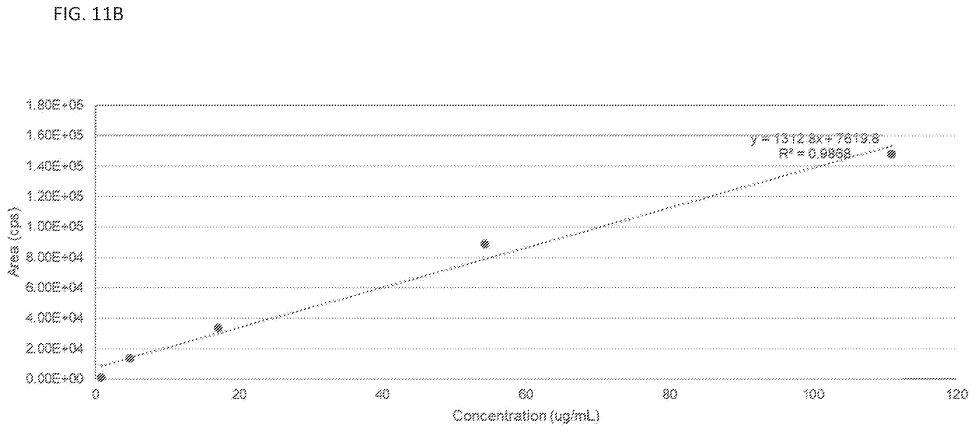

Calibration curves for target analyte gabapentin are prepared for sample volumes of 50 nL, over a concentration range of 0 to about 110 μg/mL). The calibration curves are prepared with internal standard (ritalinic acid), with the x-axis as the gabapentin concentration, and the y-axis as either the peak ratio of gabapentin vs. ritalinic acid or peak area (in cps). As illustrated by FIGS. 11A and 11B, the calibration curves demonstrate good linearity over the calibration concentrations.

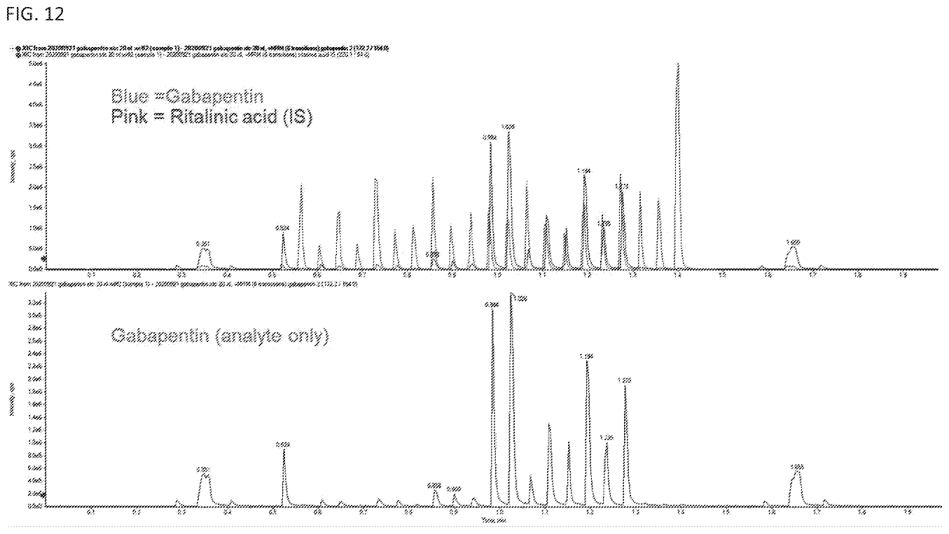

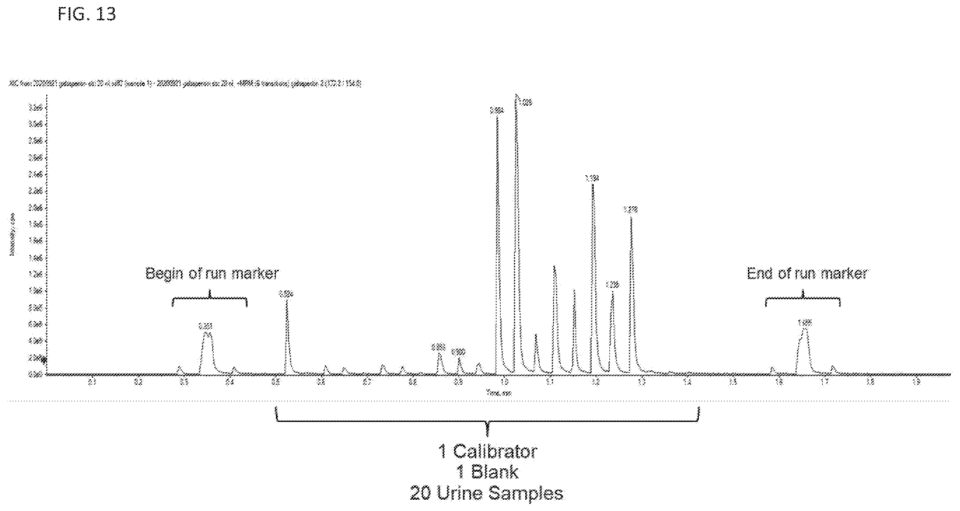

Samples (20 nL) are ejected by ADE into the open port interface operably connected to an ESI ion source, operated in MRM mode (220.1/84.0 (ritalinic acid detection) and 172.2/154.0 for gabapentin analysis). Total run time between the start of run and end of run markers is 53.16 seconds. See, FIGS. 12, 13.

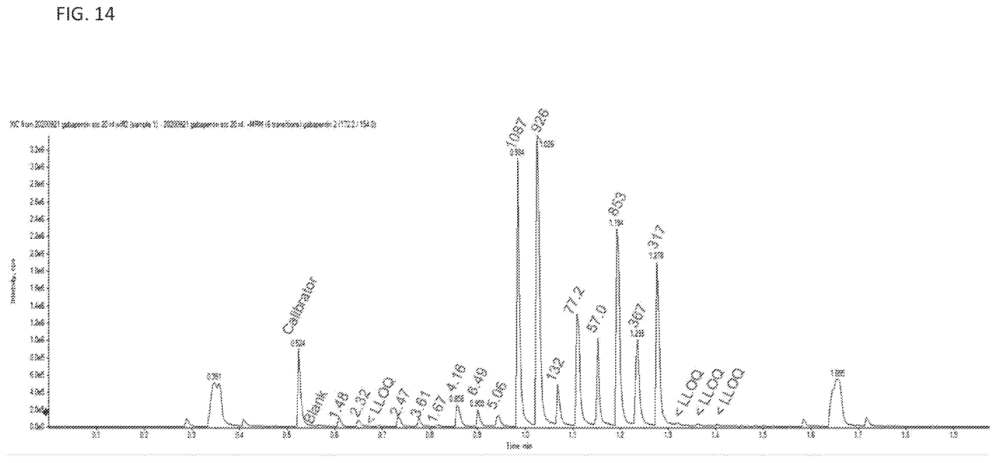

Based on the XIC intensities and established calibration curves, an estimation of gabapentin concentration in each sample can be made. Further confirmatory definitive testing of each urine sample is made by LC/MS using a Microflow M5 column (50×1 mm, 2.6 um) on a 6500+ QTRAP (Sciex). The definitive analytical testing confirms that the high throughput analytical methods provide good estimates of target analyte concentrations, as shown in FIG. 14.

### Example 2: Single Tube Calibrations

Assay throughput capacity can be increased by reducing the time needed to calibrate an assay calibration. As illustrated below, a single tube calibrator that comprises a mix of isotopologues of the target analyte, or another calibrator, that are present at different concentrations in a solution can provide good calibration and reduce overall assay time by replacing traditional, multiple-tube calibrators (each at different concentrations). A single sampling/injection from a single-tube calibrator can generate a full calibration curve such that each calibration point is from the multiple reaction monitoring (MRM) signal corresponding to a specific isotopologue.

A series of single tube calibration experiments are performed for fentanyl, norfentanyl, and gabapentin that can generate at least six calibration points from a single ejection of prepared calibrator mixture.

### Fentanyl.

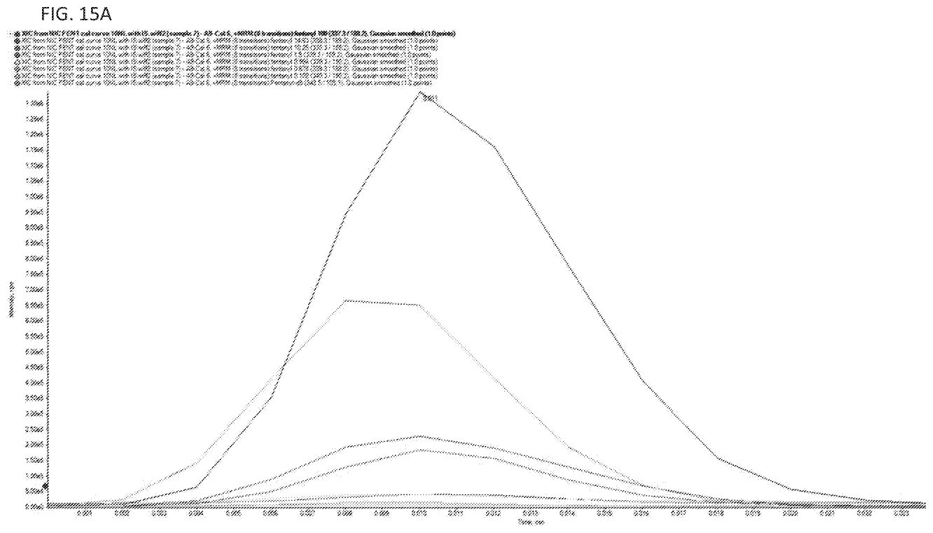

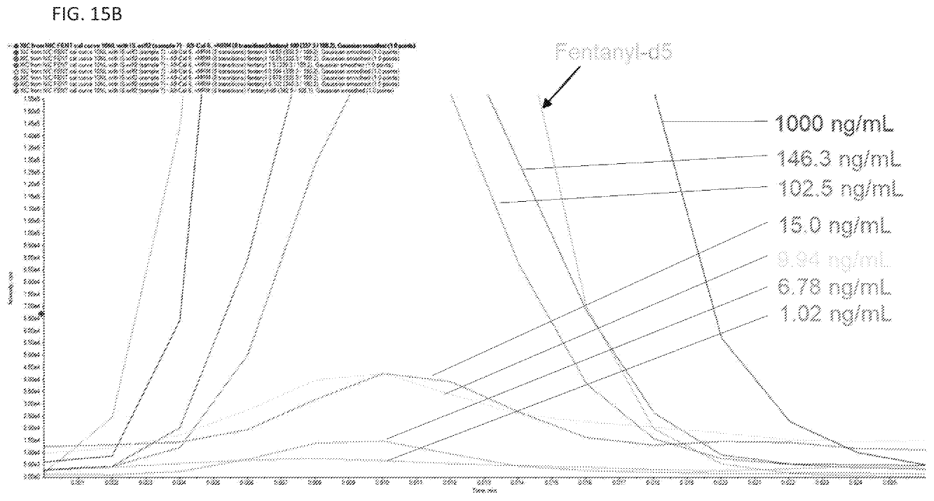

As illustrated in FIGS. 15A-15B, fentanyl can be effectively calibrated using a single tube natural isotope calibrator composition. A single 10 nL injection is effective to monitor a total of 8 MRM transitions, with concentrations at 1000 ng/ml (337.3/188.2), 146.3 ng/ml (338.3/189.2), 102.5 ng/ml (338.3/188.2), 15.0 ng/ml (339.3/189.2), 9.94 ng/mL (339.3/190.2), 6.78 ng/ml (339.3/188.2), and 1.02 ng/mL (340.3/190.2). Fentanyl-d5 signal is monitored for the transition 342.5/105.1.

### Norfentanyl.

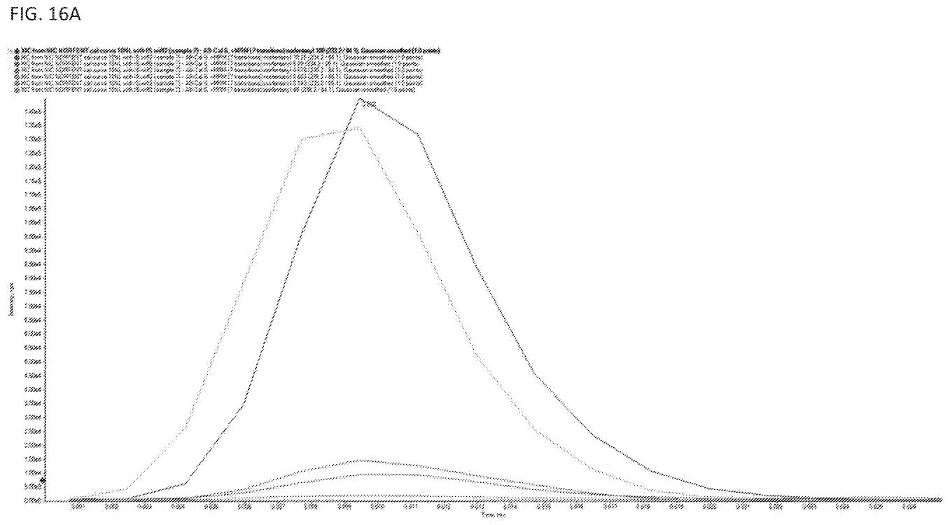

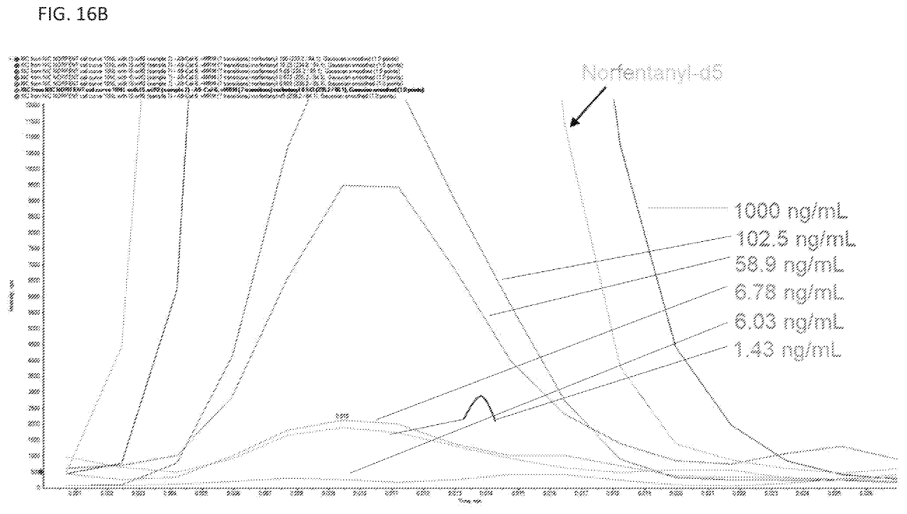

As illustrated in FIGS. 16A-16B, norfentanyl can be effectively calibrated using a single tube natural isotope calibrator composition. A single 10 nL injection is effective to monitor a total of 7 MRM transitions, with concentrations at 1000 ng/ml (233.2/84.1), 102.5 ng/ml (234.2/84.1), 58.9 ng/ml (234.2/85.1), 6.78 ng/ml (235.2/84.1), 6.03 ng/ml (235.2/85.1), and 1.43 ng/mL (235.2/86.1). Norfentanyl-d5 signal is monitored for the transition 238.2/84.1.

### Gabapentin.

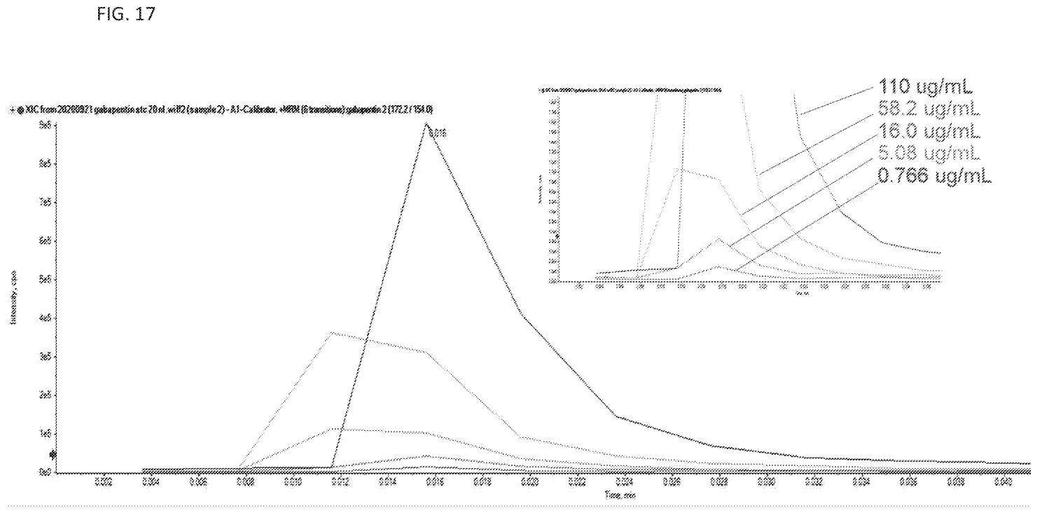

As illustrated in FIG. 17, gabapentin can be effectively calibrated using a single tube natural isotope calibrator composition. A single 20 nL injection is effective to monitor a total of 6 MRM transitions, with concentrations with concentrations at 110 ug/mL, 58.2 ug/mL, 16.0 ug/mL, 5.08 ug/mL, and 0.766 ug/mL, with primary signal monitored for the transition 172.2./154.0.

### Fentanyl & Norfentanyl Overlayed Single Tube Calibrators.

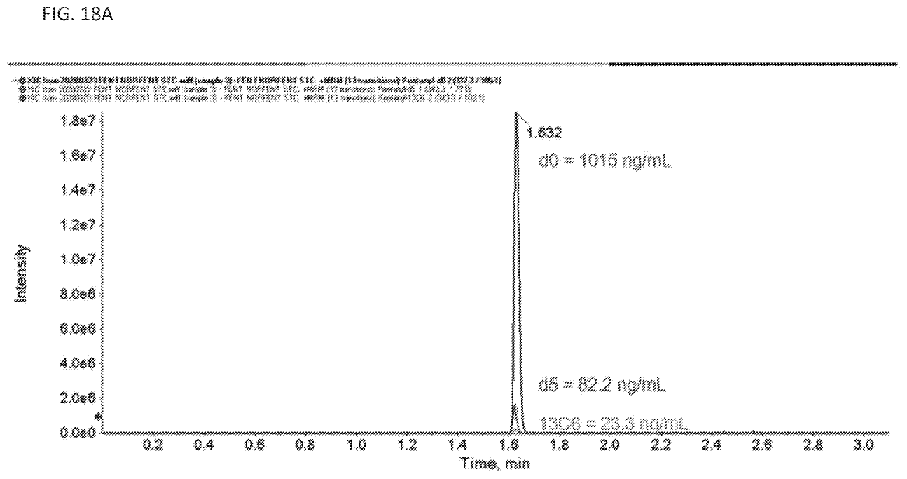

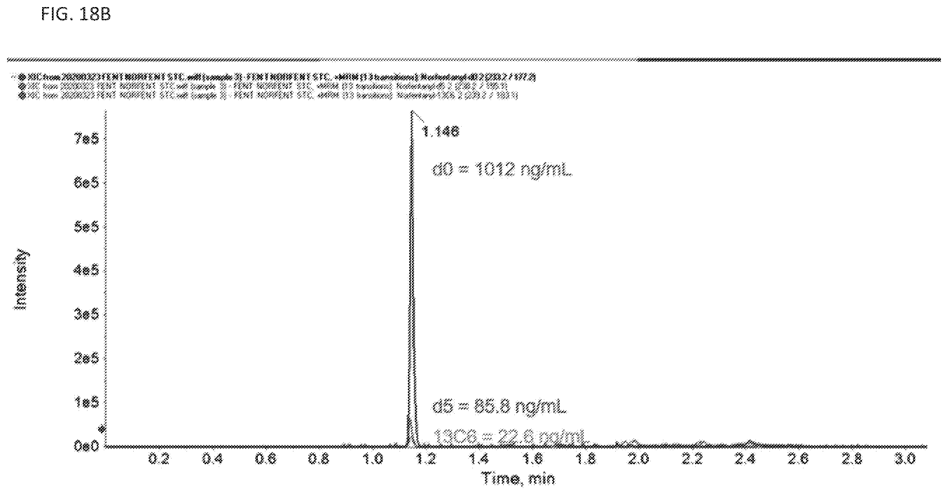

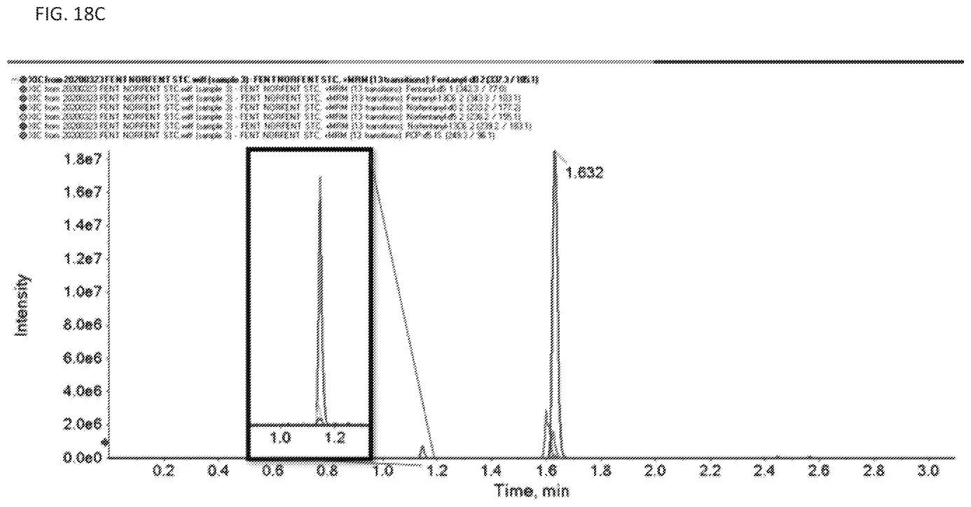

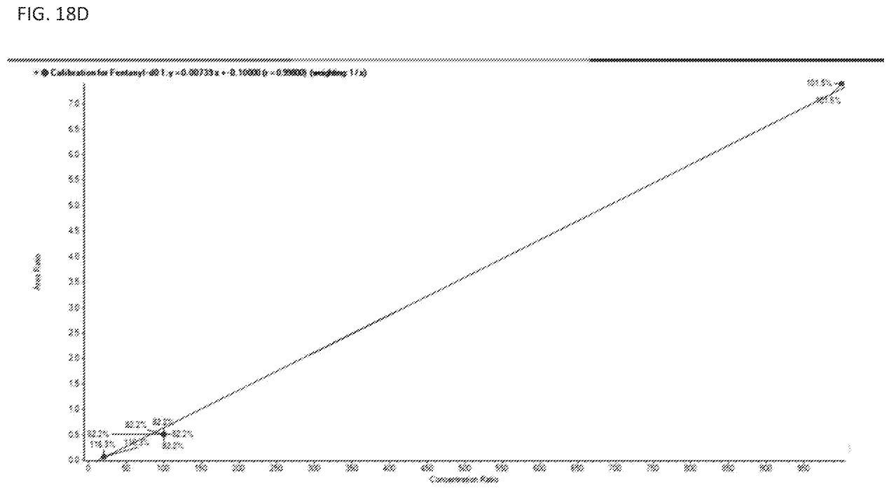

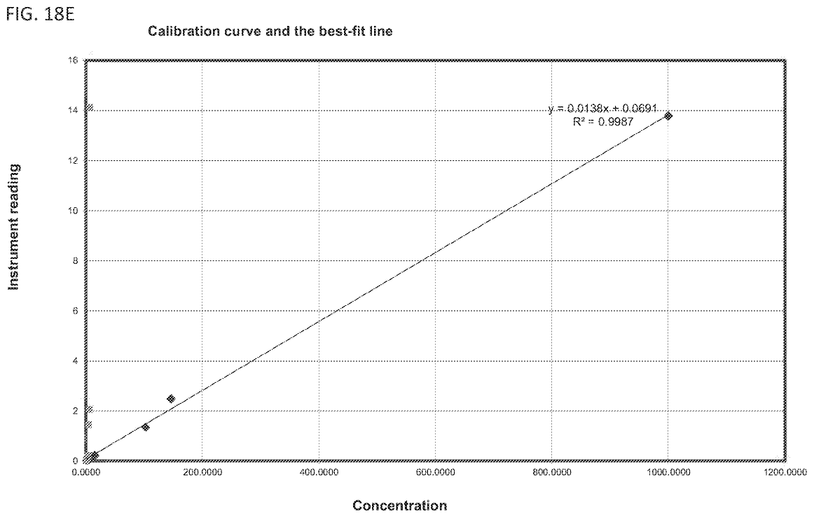

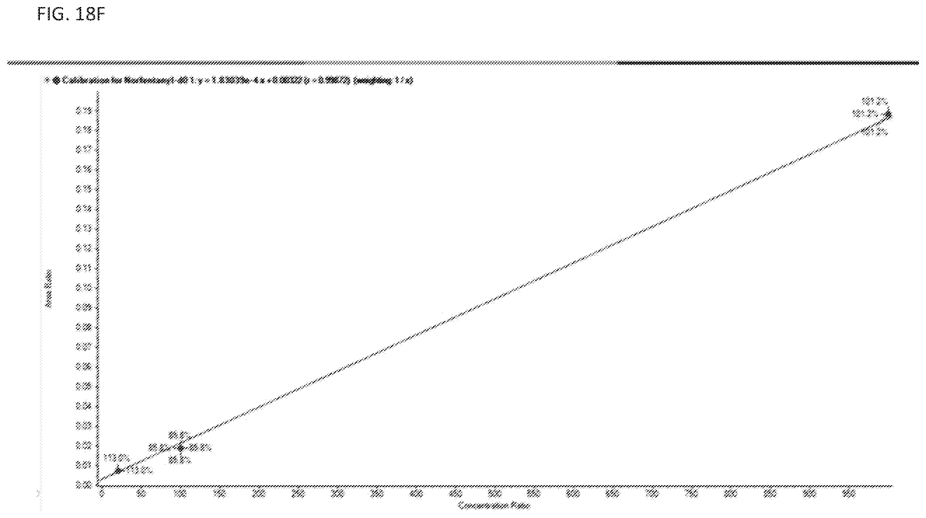

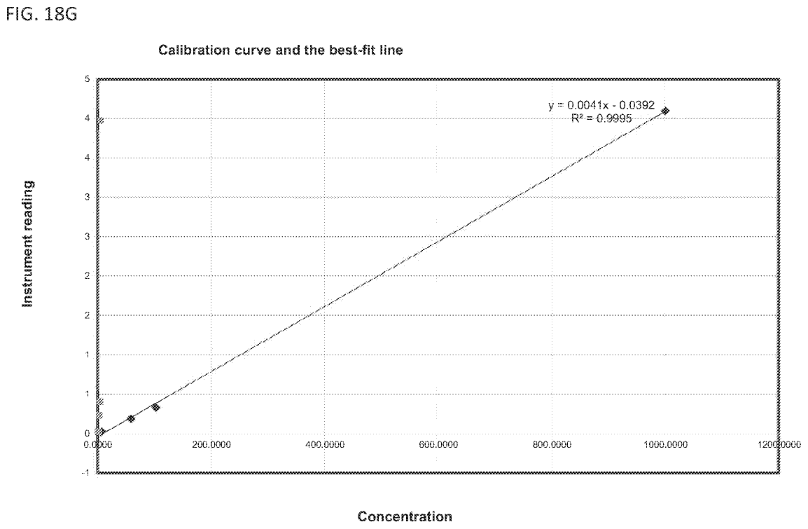

Separate single tube calibrators comprising fentanyl (at 1015 ng/ml) with fentanyl-d5 (82.2 ng/mL) and fentanyl-¹³C6 (23.3 ng/mL) isotopologues and another comprising norfentanyl (1012 ng/mL) with norfentanyl-d5 (85.8 ng/mL) and norfentanyl-¹³C6 (22.6 ng/ml) isotopologues are effective to calibrate both fentanyl and norfentanyl. Single droplets (10 nL) are ejected and a total of 13 MRM transitions are monitored with fentanyl (d0) transition at (337.3/105.1), fentanyl-d5 at (342.3/77.0), and fentanyl-¹³C6 (343.3/103.1), and the norfentanyl (d0) transition at (233.2/177.2), norfentanyl-d5 at (238.2/155.1), and norfentanyl-¹³C6 (239.2/183.1). Fentanyl-d5 signal is monitored for the transition 342.5/105.1. See FIGS. 18A-18C. The resulting calibration curves (single tube by LC-MS/MS 18D, 18F, and natural isotope by Echo MS, 18E, 18G) are plotted with R values for fentanyl in FIGS. 18D (R=0.9980), 18E (R²=0.9987) and for norfentanyl in FIGS. 18F (R=9987), 18G (R²=0.9995), with y-axis units in area ratio and x-axis in ng/ml.

As illustrated above, the examples demonstrate that single tube calibration can decrease overall assay run time and reduce waste. A single tube calibration uses only one sample reservoir/sample well, instead of multiple reservoirs/wells, which can increase assay throughput (more samples per batch). Further single tube calibrators can be more analytically accurate than traditional calibration since there is no variation between different calibrator compositions.

The illustrative examples also demonstrate methods and systems providing high throughput mass spec analysis can rapidly and accurately screen for specific target analytes in complex samples (e.g., environment, biological sample), while also providing an accurate quantification of the amount of target analyte in the sample. The results provided by the high throughput methods in accordance with the various aspects and embodiments of the disclosure can be confirmed by subsequent quantitative analytic methods.

1. A method for detecting and quantifying a target analyte in a sample by mass analysis, the method comprising:
providing the sample in a sample reservoir;
ejecting a volume of the sample into a sampling interface;
transferring the volume of sample from the sampling interface to a mass spectrometer for mass analysis,
detecting by a first analysis by mass spectrometry the presence of ions of the specific analyte in the sample, and when said ions of the specific analyte are detected, performing a second analysis that quantifies the amount of the specific analyte present in the sample.

2. (canceled)

3. The method according to claim 1, wherein the second analysis comprises ionizing the sample to form ions of the target analyte and quantifying the ions of the target analyte by mass spectrometry.

4. The method according to claim 1, wherein the sample comprises a biological sample.

5. The method according to claim 1, further comprising preparing the sample for analysis.

6. The method according to claim 5, wherein preparing the sample comprises diluting the sample with a labelled internal standard.

7. The method according to claim 5, wherein preparing the sample comprises desalting the sample.

8. The method according to claim 5, wherein preparing the sample comprises solid phase micro extraction (SPME).

9. The method according to claim 1, wherein the first analysis, second analysis, or both, includes differential mobility spectrometry (DMS).

10. The method of claim 9, wherein one of the first analysis or second analysis is conducted with the DMS set to transparent mode and the other analysis is conducted with the DMS separation voltage turned on to select a population of ions.

11. The method according to claim 1, wherein the target analyte comprises a drug of abuse, or metabolite thereof.

12. The method according to claim 1, wherein the target analyte comprises an opioid, or metabolite thereof.

13. The method according to claim 1, wherein the target analyte comprises fentanyl, norfentanyl, gabapentin, pregabalin, or PCP.

14. The method according to claim 1, wherein the at least one calibration standard comprises two or more different calibration concentrations.

15. The method according to claim 1, wherein quantifying the target analyte ions present in the sample is based on a calibration curve generated from the at least one calibration standard.

16. The method according to claim 1, wherein the limit of quantitation (LOQ) of the analyte is determined.

17. The method according to claim 1, wherein the method comprises a plurality of samples.

18. The method according to claim 1, wherein the method comprises ejecting sample at a rate of about one sample per second.

19. The method according to claim 3, wherein the second analysis is performed by liquid chromatography mass spectrometry (LC-MS).

20. The method according to claim 3 wherein the second analysis comprises ejecting by ADE the at least one calibration standard into a mobile phase capture fluid at an OPI; detecting ions of the calibration standard by mass spectrometry; generating calibration data; ejecting by ADE the sample into the mobile phase capture fluid at the OPI; and (i) detecting for the presence of target analyte ions in the sample by mass analysis, and (ii) quantifying the target analyte ions based on the calibration data.

21. The method according to claim 1, wherein the one or more calibration standard and the at least one sample are in the same sample reservoir.

22. A method for detecting and quantifying a target analyte in a sample by mass analysis, the method comprising:
providing a sample plate comprising a plurality of sample reservoirs, wherein at least one sample reservoir comprises at least one calibration standard for the target analyte, and at least one sample reservoir comprises the sample;
acoustically ejecting the at least one sample into a mobile phase at an open port interface (OPI) using an acoustic droplet ejector (ADE);
ionizing the at least one sample; and
detecting by a first analysis by mass spectrometry the presence of ions of the target analyte in the sample, and when said ions of the target analyte are detected, performing a second analysis that quantifies the amount of the target analyte present in the sample.

23. The method according to claim 22, wherein the second analysis comprises ionizing the sample to form ions of the target analyte and quantifying the ions of the target analyte by mass spectrometry.

24. The method according to claim 22, wherein the sample comprises a biological sample.

25. The method according to claim 22, further comprising preparing the sample for analysis.

26. The method according to claim 25, wherein preparing the sample comprises diluting the sample with a labelled internal standard.

27. The method according to claim 25, wherein preparing the sample comprises desalting the sample.

28. The method according to claim 25, wherein preparing the sample comprises solid phase micro extraction (SPME).

29. The method according to claim 22, wherein the first analysis, second analysis, or both, includes differential mobility spectrometry (DMS).

30. The method according to claim 29, wherein one of the first analysis or second analysis is conducted with the DMS set to transparent mode and the other analysis is conducted with the DMS separation voltage turned on to select a population of ions.

31. The method according to claim 22, wherein the target analyte comprises a drug of abuse, or metabolite thereof.

32. The method according to claim 22, wherein the target analyte comprises an opioid, or metabolite thereof.

33. The method according to claim 22, wherein the target analyte comprises fentanyl, norfentanyl, gabapentin, pregabalin, or PCP.

34. The method according to claim 22, wherein the at least one calibration standard comprises two or more different calibration concentrations.

35. The method according to claim 22, wherein quantifying the target analyte ions present in the sample is based on a calibration curve generated from the at least one calibration standard.

36. The method according to claim 22, wherein the limit of quantitation (LOQ) of the analyte is determined.

37. The method according to claim 22, wherein the method comprises a plurality of samples.

38. The method according to claim 22, wherein the method comprises ejecting sample at a rate of about one sample per second.

39. The method according to claim 23, wherein the second analysis is performed by liquid chromatography mass spectrometry (LC-MS).

40. The method according to claim 23, wherein the second analysis comprises ejecting by ADE the at least one calibration standard into a mobile phase capture fluid at an OPI; detecting ions of the calibration standard by mass spectrometry; generating calibration data; ejecting by ADE the sample into the mobile phase capture fluid at the OPI; and (i) detecting for the presence of target analyte ions in the sample by mass analysis, and (ii) quantifying the target analyte ions based on the calibration data.

41. The method according to claim 22, wherein the one or more calibration standard and the at least one sample are in the same sample reservoir.
